SEQUENCE LISTING (44 sequences)

SEQ ID NO: 1 - DNA, 2211 bp, synthetic construct
  misc_feature 1..2211: note = AAV9 hu.14 coding sequence with ACG start codon
  source 1..2211: mol_type = other DNA
acggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 2 - DNA, 2211 bp, synthetic construct
  misc_feature 1..2211: note = AAV9 hu.14 coding sequence with ACG start codon and silentmutations in codons 7 and 8
  source 1..2211: mol_type = other DNA
acggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3 - DNA, 2721 bp, synthetic construct
  misc_feature 1..2721: note = VD919 Cap9 expression cassette
  misc_feature 1..260: note = Polyhedrin promoter long
  misc_feature 261..269: note = Kozak sequence
  misc_feature 270..2480: note = ORF
  variation 288..293: note = mutation - silent mutations CTTCCA to CTACCC
  regulatory 2481..2721: note = polyA_signal - SV40-polyA; regulatory_class = polyA_signal_sequence
  source 1..2721: mol_type = other DNA
tgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccacggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 4 - DNA, 2721 bp, synthetic construct
  misc_feature 1..2721: note = VD920 Cap9 expression cassette
  misc_feature 1..260: note = Polyhedrin promotor long
  misc_feature 261..269: note = Kozak sequence
  misc_feature 270..2480: note = ORF
  variation 288..293: note = mutation - silent mutations CTTCCA to CTACCC
  regulatory 2481..2721: regulatory_class = polyA_signal_sequence
  source 1..2721: mol_type = other DNA
tgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccctggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 5 - DNA, 2611 bp, synthetic construct
  misc_feature 1..2611: note = VD921 Cap9 expression cassette
  misc_feature 1..159: note = Polyhedrin promotor short
  misc_feature 160..2370: note = ORF
  variation 178..183: note = mutation - silent mutations CTTCCA to CTACCC
  regulatory 2371..2611: regulatory_class = polyA_signal_sequence
  source 1..2611: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatacggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 6 - DNA, 2611 bp, synthetic construct
  misc_feature 1..2611: note = VD922 Cap9 expression cassette
  misc_feature 1..159: note = Polyhedrin promotor short
  misc_feature 160..2370: note = ORF
  variation 178..183: note = mutation - silent mutations CTTCCA to CTACCC
  regulatory 2371..2611: regulatory_class = polyA_signal_sequence
  source 1..2611: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 7 - DNA, 2614 bp, synthetic construct
  misc_feature 1..2614: note = VD923 Cap9 expression cassette
  misc_feature 1..159: note = Polyhedrin promotor short
  misc_feature 160..2373: note = ORF
  misc_feature 163..165: note = extra Ala codon
  variation 181..186: note = mutation - silent mutations CTTCCA to CTACCC
  regulatory 2374..2614: regulatory_class = polyA_signal_sequence
  source 1..2614: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatacggctgctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 8 - DNA, 2614 bp, synthetic construct
  misc_feature 1..2614: note = VD924 Cap9 expression cassette
  misc_feature 1..159: note = Polyhedrin promotor short
  misc_feature 160..2373: note = ORF
  misc_feature 163..165: note = extra Ala codon
  variation 181..186: note = mutation - silent mutations CTTCCA to CTACCC
  regulatory 2374..2614: regulatory_class = polyA_signal_sequence
  source 1..2614: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggctgctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 9 - DNA, 250 bp, synthetic construct
  misc_feature 1..250: note = polH promoter long
  source 1..250: mol_type = other DNA
tgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagctt

SEQ ID NO: 10 - DNA, 155 bp, synthetic construct
  misc_feature 1..155: note = polH promoter short
  source 1..155: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaat

SEQ ID NO: 11 - skipped

SEQ ID NO: 12 - protein, 736 aa, Adeno-associated virus
  source 1..736: note = 9 isolate hu.14
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 738 aa, Adeno-associated virus
  source 1..738: note = isolate rh.10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 14 - DNA, 2217 bp, synthetic construct
  misc_feature 1..2217: note = AAVrh.10 coding sequence with ACG start codon
  source 1..2217: mol_type = other DNA
acggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 15 - DNA, 2211 bp, synthetic construct
  misc_feature 1..2211: note = AAV serotype 1 coding sequence with ACG start codon
  source 1..2211: mol_type = other DNA
acggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 16 - DNA, 2211 bp, synthetic construct
  misc_feature 1..2211: note = AAV serotype 6 coding sequence with ACG start codon
  source 1..2211: mol_type = other DNA
acggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcacgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 17 - DNA, 2214 bp, synthetic construct
  misc_feature 1..2214: note = AAV serotype 7 coding sequence with ACG start codon
  source 1..2214: mol_type = other DNA
acggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 18 - DNA, 2217 bp, synthetic construct
  misc_feature 1..2217: note = AAV serotype 8 coding sequence with ACG start codon
  source 1..2217: mol_type = other DNA
acggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 19 - DNA, 1194 bp, synthetic construct
  misc_feature 1..1194: note = Rep52 wt
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 20 - DNA, 1194 bp, synthetic construct
  misc_feature 1..1194: note = rep52 sf9 (insect cell) optimised
  source 1..1194: mol_type = other DNA
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcaggaggtgaaggacttcttccgttgggctaaggaccacgtggtggaggtggagcacgagttctacgtgaagaagggtggtgctaagaagcgtcctgctccttccgacgctgacatctccgagcctaagcgtgtgcgtgagtccgtggctcagccttccacctccgacgctgaggcttccatcaactacgctgaccgttaccagaacaagtgctcccgtcacgtgggtatgaacctgatgctgttcccttgccgtcagtgcgagcgtatgaaccagaactccaacatctgcttcacccacggtcagaaggactgcctggagtgcttccctgtgtccgagtcccagcctgtgtccgtggtgaagaaggcttaccagaagctgtgctacatccaccacatcatgggtaaggtgcctgacgcttgcaccgcttgcgacctggtgaacgtggacctggacgactgcatcttcgagcagtaa

SEQ ID NO: 21 - DNA, 1194 bp, synthetic construct
  misc_feature 1..1194: note = rep52 AT optimised
  source 1..1194: mol_type = other DNA
atggaattagtaggatggttagtagataaaggaataacatcagaaaaacaatggatacaagaagatcaagcatcatatatatcatttaatgcagcatcaaattcaagatcacaaataaaagcagcattagataatgcaggaaaaataatgtcattaacaaaaacagcaccagattatttagtaggacaacaaccagtagaagatatatcatcaaatagaatatataaaatattagaattaaatggatatgatccacaatatgcagcatcagtatttttaggatgggcaacaaaaaaatttggaaaaagaaatacaatatggttatttggaccagcaacaacaggaaaaacaaatatagcagaagcaatagcacatacagtaccattttatggatgtgtaaattggacaaatgaaaattttccatttaatgattgtgtagataaaatggtaatatggtgggaagaaggaaaaatgacagcaaaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 22 - DNA, 1194 bp, synthetic construct
  misc_feature 1..1194: note = rep52 GC optimised
  source 1..1194: mol_type = other DNA
atggagctggtggggtggctggtggacaaggggatcacgagcgagaagcagtggatccaggaggaccaggcgagctacatcagcttcaacgcggcgagcaacagccggagccagatcaaggcggcgctggacaacgcggggaagatcatgagcctgacgaagacggcgccggactacctggtggggcagcagccggtggaggacatcagcagcaaccggatctacaagatcctggagctgaacgggtacgacccgcagtacgcggcgagcgtgttcctggggtgggcgacgaagaagttcgggaagcggaacacgatctggctgttcgggccggcgacgacggggaagacgaacatcgcggaggcgatcgcgcacacggtgccgttctacgggtgcgtgaactggacgaacgagaacttcccgttcaacgactgcgtggacaagatggtgatctggtgggaggaggggaagatgacggcgaaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 23 - DNA, 1194 bp, synthetic construct
  misc_feature 1..1194: note = Extreme codon optimized Rep52
  source 1..1194: mol_type = other DNA
atggaattggtgggttggcttgtcgataaaggcataacgagcgaaaaacaatggattcaagaagatcaagcatcctatattagctttaacgctgcgagtaatagcagaagccagataaaagcagcgttggacaacgctggtaagatcatgtcgcttacaaagacggcgccggactatctcgtcggtcaacaaccagttgaggacataagttcaaacagaatttacaagatcttggagctcaatggttatgaccctcagtacgctgcgtctgtattcctcggatgggctacaaagaaatttggtaaacgcaatactatttggctcttcggtccagccacaaccggcaaaactaatattgccgaagctattgctcataccgtcccattttatggttgtgtgaattggactaacgaaaatttcccttttaatgattgcgtggataaaatggtcatatggtgggaagaaggtaaaatgacagctaaagtggttgaatccgcgaaggcaatcctgggtggttccaaagtccgtgtcgatcaaaagtgtaaaagctccgctcaaatcgatcctaccccggtaattgttacgagtaatactaatatgtgcgcagttattgacggtaactcgacgacgtttgaacatcaacaacccctccaggatagaatgtttaagtttgagctgactagacgcctcgaccacgatttcggtaaagttactaaacaggaggtgaaggatttctttcgctgggctaaagaccatgtcgtagaagtcgagcacgagttttatgttaagaaaggaggtgctaaaaagcgtcctgctcctagcgatgctgacatctccgaacctaagcgtgtccgtgaaagcgtcgcacaacctagcaccagcgatgctgaggcctccattaattatgctgatcgttatcagaataagtgcagccgccatgtcggtatgaacctcatgctcttcccttgtcgtcagtgtgaacgtatgaaccaaaactcaaacatatgttttacccatggtcaaaaggactgcctggagtgcttccctgtaagtgaatcgcaacctgtaagtgtggtaaagaaagcgtaccaaaagctctgttatatccaccacattatgggcaaagtaccggatgcctgtaccgcgtgtgaccttgtgaacgttgacctggacgattgtatattcgagcagtaa

SEQ ID NO: 24 - DNA, 1194 bp, synthetic construct; identical to SEQ ID NO: 19
  misc_feature 1..1194: note = Rep52
  source 1..1194: mol_type = other DNA

SEQ ID NO: 25 - DNA, 1873 bp, synthetic construct
  misc_feature 1..1873: note = Rep78
  source 1..1873: mol_type = other DNA
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 26 - DNA, 28 bp, synthetic construct
  misc_feature 1..28: note = hr 28-mer
  source 1..28: mol_type = other DNA
ctttacgagtagaattctacgcgtaaaa

SEQ ID NO: 27 - DNA, 3111 bp, synthetic construct
  misc_feature 1..3111: note = CMV-Seap transgene construct
  source 1..3111: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgccaaaccgcggctccagccctctccagcctccggctcagccggctcatcagtcggtcaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtccgtgttgcttggtcttcacctgtgcagaattgcgaaccatggattcatcgacggtaccgcgggccctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcccc

SEQ ID NO: 28 - DNA, 3466 bp, synthetic construct
  misc_feature 1..3466: note = NaGlu-myc transgene construct
  source 1..3466: mol_type = other DNA
tctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgagaggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacccggatcccccgggctgcaggaattccgagaccatggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactccttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattaccccggctgggtggccggctcttggggctcctccggcgaacaaaaacttatttctgaagaagatctgtgatgatagattcgccaccactgggccttgttttccgctaattccagggcagattccagggcccagagctggacagacatcacaggataacccaggcctgggaggaggccccacggcctgctggtggggtctgacctggggggattggagggaaatgacctgccctccaccaccacccaaagtgtgggattaaagtagcttggtaccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccag

SEQ ID NO: 29 - DNA, 4123 bp, synthetic construct
  misc_feature 1..4123: note = VD183 construct with Rep52 and Rep78 expression cassettes
  source 1..4123: mol_type = other DNA
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 30 - DNA, 2618 bp, synthetic construct
  misc_feature 1..2618: note = AAVrh.10 expression cassette 1580
  source 1..2618: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 31 - DNA, 2618 bp, synthetic construct
  misc_feature 1..2618: note = AAVrh.10 expression cassette 1581
  source 1..2618: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatacggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 32 - DNA, 2562 bp, synthetic construct
  misc_feature 1..2562: note = AAVrh.10 expression cassette 1582
  source 1..2562: mol_type = other DNA
acctttaattcaacccaacacaatatattatagttaaataagaattattatcaaatcatttgtatattaattaaaatactatactgtaaattacattttatttacggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 33 - DNA, 2562 bp, synthetic construct
  misc_feature 1..2562: note = AAVrh.10 expression cassette 1583
  source 1..2562: mol_type = other DNA
acctttaattcaacccaacacaatatattatagttaaataagaattattatcaaatcatttgtatattaattaaaatactatactgtaaattacattttatttctggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 34 - DNA, 2562 bp, synthetic construct
  misc_feature 1..2562: note = AAVrh.10 expression cassette 1584
  source 1..2562: mol_type = other DNA
acctttaattcaacccaacacaatatattatagttaaataagaattattatcaaatcatttgtatattaattaaaatactatactgtaaattacattcgatgcatggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 35 - DNA, 2618 bp, synthetic construct; identical to SEQ ID NO: 30
  misc_feature 1..2618: note = AAVrh.10 expression cassette 1625
  source 1..2618: mol_type = other DNA

SEQ ID NO: 36 - DNA, 2621 bp, synthetic construct
  misc_feature 1..2621: note = AAVrh.10 expression cassette 1626
  source 1..2621: mol_type = other DNA
cgcgtgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatacggctgctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 37 - DNA, 2565 bp, synthetic construct
  misc_feature 1..2565: note = AAVrh.10 expression cassette 1627
  source 1..2565: mol_type = other DNA
acctttaattcaacccaacacaatatattatagttaaataagaattattatcaaatcatttgtatattaattaaaatactatactgtaaattacattttatttacggctgctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 38 - DNA, 2565 bp, synthetic construct
  misc_feature 1..2565: note = AAVrh.10 expression cassette 1628
  source 1..2565: mol_type = other DNA
acctttaattcaacccaacacaatatattatagttaaataagaattattatcaaatcatttgtatattaattaaaatactatactgtaaattacattttatttctggctgctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 39 - DNA, 2721 bp, synthetic construct
  misc_feature 1..2721: note = AAV6 expression cassette 1083
  source 1..2721: mol_type = other DNA
tgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccctggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 40 - DNA, 2607 bp, synthetic construct
  misc_feature 1..2607: note = AAV6 expression cassette 1084
  source 1..2607: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatctggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 41 - DNA, 2721 bp, synthetic construct
  misc_feature 1..2721: note = AAV6 expression cassette 1085
  source 1..2721: mol_type = other DNA
tgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccgtggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 42 - DNA, 2607 bp, synthetic construct
  misc_feature 1..2607: note = AAV6 expression cassette 1086
  source 1..2607: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatgtggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 43 - DNA, 2721 bp, synthetic construct
  misc_feature 1..2721: note = AAV6 expression cassette 1087
  source 1..2721: mol_type = other DNA
tgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccacggctgccgatggttatctacccgattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 44 - protein, 736 aa, synthetic construct
  REGION 1..224: note = AAV6 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

(19) United States
(12) Patent Application Publication
(10) Pub. No.: US 2025/0297278 A1
(43) Pub. Date: Sep. 25, 2025

(54) Insect cell-produced high potency AAV vectors with CNS-tropism

(71) Applicant: uniQure biopharma B.V, Amsterdam (NL)

(72) Inventors: David Johannes Francois Du Plessis, Amsterdam (NL); Sebastiaan Menno Bosma, Amsterdam (NL); - Anggakusuma, Amsterdam (NL)

(73) Assignee: uniQure biopharma B.V., Amsterdam (NL)

(21) Appl. No.: 18/683,254

(22) PCT Filed: Aug. 26, 2022
(86) PCT No.: PCT/EP2022/073738

§ 371 Date: Feb. 13, 2024

(30) Foreign Application Priority Data
Aug. 26, 2021 (EP) 21193344.5

(51) Int. Cl. C12N 15/86; C07K 14/005

(52) U.S. Cl. CPC C12N 15/86; C07K 14/005; C12N 2750/14122; C12N 2750/14143; C12N 2750/14152

(57) ABSTRACT

The present invention pertains to nucleic acid constructs for expression of capsid protein of neurotropic AAV vectors in insect cells that allow the manufacture of such AAV vectors with improved potency. The invention further elates to insect cells comprising such constructs and method wherein the insect are used for the production neurotropic AAV vectors with high potency.

### REFERENCE TO AN ELECTRONIC SEQUENCE LISTING

The contents of the electronic sequence listing (P6104516PCT-US_20240716_Sequence Listing.xml; Size: 152 Kb; and Date of Creation: Jul. 16, 2024) is herein incorporated by reference in its entirety.

### FIELD OF THE INVENTION

The present invention relates to the fields of molecular virology and gene therapy. In particular the invention relates to means and methods for producing neurotropic AAV vectors in insect cells that have a higher potency than the corresponding vectors produced in mammalian cells.

### BACKGROUND OF THE INVENTION

Recombinantly produced adeno-associated virus (AAV) is widely used as a vector in gene therapy of humans. The AAV capsid, that packages the therapeutic DNA to be delivered, consists of three capsid proteins, VP1, VP2, and VP3, the natural biosynthesis of which involves alternative splicing and differential start codon usage from a single capsid open reading frame (ORF) in the AAV genome. The VP3 amino acid sequence is common between all three capsid proteins, whereas VP2 and VP1 have longer N-terminal sequences. The unique part of VP N-terminal sequence contains a phospholipase A2 domain that is critical for the virus' infectivity.

The relative amounts of VP1/P2NP3 in naturally produced AAV are generally estimated to be 1/1/10. Importantly, for recombinantly produced AAV (rAAV) there appears to be no fixed VP1NP2NP3 stoichiometry. Rather, the assembly is stochastic such that the relative amounts of VP1NP2NP3 that are incorporated in the capsid depend mainly on their relative expression levels in a given host cell (Snijder et al., 2014, J. Am. Chem. Soc. 136: 7295-7299). Consequently, the design of the expression vectors for the capsid proteins is essential for the biological potency of the AAV vectors produced in a given system.

There are currently two types of production systems in use for the production of clinical grade AAV vectors: mammalian (HEK293) cell-based systems and insect cell systems, the latter mostly based on using at least one baculoviral expression vector (BEV). The insect cell-based systems for manufacturing AAV offer several advantages over mammalian cell-based rAAV systems, including scalability of non-adherent cells, cost savings due to the use of serum-free growth conditions and no need for adenoviral helper functions. However, most of the AAV serotypes produced in this system suffer from lower transduction efficiencies compared with HEK293-derived AAV vectors because of a suboptimal content of VP1 capsid protein and its essential phospholipase A2 activity.

The original insect cell system used a non-canonical ACG initiation codon for VP1 to induce leaky ribosome scanning for expression of AAV serotype 2 (AAV2) capsids (Urabe et al., 2002, Hum. Gene Ther. 13: 1935-1943) and AAV2/5 chimeric capsids (US 2004/197895). However, for other serotypes, e.g. AAV5 and AAV8, the use of an ACG initiation codon for VP1 resulted in AAV vectors with a reduced potency due to an insufficient amount of VP1 (Kohlbrenner et al., 2005, Mol. Ther. 12: 1217-1225; Urabe et al., 2006 J. Virol. 80: 1874-1885; and Mietzsch et al., 2015, Hum. Gene Ther. 26: 688-697).

WO 2021/123122 discloses constructs for expression of AAV8 capsids in plant cells using an ACG initiation codon for a VP1 coding sequence that is operably linked to a the CaMV35S promoter.

WO 2021/113767 discloses constructs for expression of AAV capsid proteins in insect cells. While non-canonical initiation codons are used to reduce expression of chimeric AAV6/2/9 VP1s, expression of VP2 and VP3 does not rely on leaky scanning of the VP1 coding sequence. Instead, VP2 and VP3 proteins are expressed from a separate expression cassette, while the VP2 and VP3 initiation codons in the VP1 coding sequence are inactivated to ensure that translation of the VP1 coding sequence in an insect cell produces only VP1 but not the VP2 and VP3 capsid proteins.

US 2020/0248206 discloses that AAV5 capsids can be efficiently produced in insect cells from an expression construct encoding a transcript for the VP1, VP2, and VP3 proteins from overlapping reading frames, wherein VP1 is translated from an AUG initiation codon.

Kurasawa et al. (2020, Mol Ther Methods Clin Dev, 19: 330-340) confirm that also for insect cell-produced AAV9 vectors, the use of ACG as VP1 initiation codon in combination with a p10 promoter results in an AAV9 vector having a reduced in vivo transduction efficiency as compared to the mammalian cell-derived AAV9 vector.

For AAV5 these problems have been successfully addressed using a combination of CUG as suboptimal VP1 start codon and an extra alanine codon inserted immediately after the start codon (WO2015/137802) or by utilising an attenuated Kozak sequence in combination with an AUG VP1 start codon to fine tune the leaky scanning of the AUG codon (Kondratov et al., 2017, Mol Ther. 25: 2661-75). However, while the latter approach produced a significantly higher biological potency of the AAV5 vector, even in a comparison with HEK293-manufactured AAV5, mediating a 4-fold higher transduction of brain tissues in mice, for the AAV9 vector there was no significant difference between the insect cell- and HEK293-manufactured AAV9 samples (Kondratov et al., 2017, supra).

There is, therefore, still a need in the art for means and methods for producing insect cell-derived AAV vectors with CNS tropism, such as AAV9 and AAV10rh (also referred to as AAV-rh10, AAV-RH10 or AAV10RH or variants thereof), that have an improved potency as compared to the corresponding mammalian cell derived vectors. Thus it is an object of the present invention to provide for such means and methods.

### SUMMARY OF THE INVENTION

In a first aspect, the invention relates to a nucleic acid construct comprising an expression cassette comprising a promoter that is active in insect cells, operably linked to a nucleotide sequence encoding an mRNA, comprising an open reading frame translation of which in an insect cell produces AAV VP1, VP2, and VP3 capsid proteins, wherein the open reading frame comprises: a) an ACG codon as suboptimal VP1 translation initiation codon; and, b) a nucleotide sequence that has at least 95% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon, and wherein the promoter that is active in insect cells is a promoter other than a baculoviral p10 promoter. In one embodiment, the promoter that is active in insect cells is a baculoviral polH promoter, preferably a polH promoter of Autographa californica nuclear polyhedrosis virus. In one embodiment, the baculoviral polH promoter comprises or consists of the nucleotide sequence in SEQ ID NO: 9 or 10, of which SEQ ID NO: 10 is most preferred.

In one embodiment, a nucleic acid construct according to the invention is a nucleic acid construct comprising an expression cassette comprising a baculoviral polH promoter, operably linked to a nucleotide sequence encoding an mRNA, comprising an open reading frame translation of which in an insect cell produces AAV VP1, VP2, and VP3 capsid proteins, wherein the open reading frame comprises: a) an ACG codon as suboptimal VP1 translation initiation codon; and, b) a nucleotide sequence that has at least 95% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon, and wherein the open reading frame encodes an amino acid sequence that has at least 85, 86, 88, 90, 92, 94, 96, 98, 99 or 100%% sequence identity with SEQ ID NO: 12, or wherein the open reading frame has at least 78, 79, 80, 81, 82, 84, 86, 88, 90, 92, 94, 96, 98, 99 or 100% nucleotide sequence identity with SEQ ID NO: 1.

In one embodiment, a nucleic acid construct according to the invention is a nucleic acid construct comprising an expression cassette comprising a baculoviral polH promoter, operably linked to a nucleotide sequence encoding an mRNA, comprising an open reading frame translation of which in an insect cell produces AAV VP1, VP2, and VP3 capsid proteins, wherein the open reading frame comprises: a) an ACG codon as suboptimal VP1 translation initiation codon; and, b) a nucleotide sequence that has at least 95% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon, and wherein the open reading frame encodes an amino acid sequence that has at least 85, 86, 87, 88, 89, 90, 92, 93, 94, 96, 98, 99 or 100% amino acid identity with SEQ ID NO: 13, or wherein the open reading frame has at least 80, 81, 82, 83, 84, 85, 86, 87, 88, 89, 90, 92, 94, 96, 98, 99 or 100% nucleotide sequence identity with SEQ ID NO: 14.

In one embodiment, a nucleic acid construct according to the invention is a nucleic acid construct comprising an expression cassette comprising a baculoviral polH promoter, operably linked to a nucleotide sequence encoding an mRNA, comprising an open reading frame translation of which in an insect cell produces AAV VP1, VP2, and VP3 capsid proteins, wherein the open reading frame comprises: a) an ACG codon as suboptimal VP1 translation initiation codon; and, b) a nucleotide sequence that has at least 95% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon, and wherein the open reading frame encodes an amino acid sequence that has at least 84, 85, 86, 87, 88, 89, 90, 92, 93, 94, 96, 98, 99 or 100% amino acid identity with SEQ ID NO: 44, or wherein the open reading frame has at least 80, 81, 82, 83, 84, 85, 86, 87, 88, 89, 90, 92, 94, 96, 98, 99 or 100% nucleotide sequence identity with SEQ ID NO: 16.

In one embodiment, a nucleic acid construct according to the invention is a nucleic acid construct wherein the ACG suboptimal VP1 translation initiation codon is not comprised in a Kozak consensus sequence that surrounds the initiation codon and wherein the ACG suboptimal VP1 translation initiation codon is not comprised in a VP2 initiator context.

In one embodiment, a nucleic acid construct according to the invention is a nucleic acid construct wherein the ACG suboptimal VP1 translation initiation codon is directly linked to the 3′ end of the promoter sequence. In one embodiment, a nucleic acid construct according to the invention is a nucleic acid construct wherein the ACG suboptimal VP1 translation initiation codon is directly linked to the 3′ end of the promoter sequence of SEQ ID NO: 9 or 10, of which SEQ ID NO: 10 is most preferred.

In one embodiment, a nucleic acid construct according to the invention is a nucleic acid construct wherein the nucleotide sequence in b) comprises at least one of i) a CTA codon in positions corresponding to position 19-21 of SEQ ID NO: 1; and ii) a CCC codon in positions corresponding to position 22-24 of SEQ ID NO: 1.

In one embodiment, a nucleic acid construct according to the invention is a nucleic acid construct wherein the amino acid sequences of the AAV VP1, VP2, and VP3 capsid proteins are comprised in an amino acid sequence of a Genbank accession number selected from the group consisting of: MT162432.1, MT162431.1, MT162430.1, MT162429.1, MT162428.1, MT162427.1, MT162426.1, MT162425.1, MT162424.1, MT162423.1, MT162422.1, MN428627.1, MN365014.1, MK163936.1, MF187357.1, MF187356.1, KT984498.1, KU056476.1, KU056475.1, KU056474.1, KU056473.1, KT235812.1, KT235811.1, KT235810.1, KT235809.1, KT235808.1, KT235807.1, KT235806.1, KT235805.1, KT235804.1, EU368926.1, EU368925.1, EU368924.1, EU368923.1, EU368922.1, EU368921.1, EU368920.1, EU368919.1, EU368918.1, EU368914.1, EU368913.1, EU368911.1, EU368910.1, EU368909.1, DQ180605.1, DQ180604.1, AY530621.1, AY530611.1, AY530601.1, AY530582.1, AY530579.1, AY530574.1, AY530572.1, AY530571.1, AY530569.1, AY530568.1, AY530566.1, AY530565.1, AY530563.1, AY530562.1, AY530560.1, AY530557.1, AY530556.1, AY243023.1, AY243022.1, AY243020.1, AY243019.1, AY243018.1, AY243016.1, AY243015.1, AY243014.1, AY243013.1, AY243011.1, AY243010.1, AY243009.1, AY243008.1, AY243006.1, AY243005.1, AY243004.1, AY243000.1, AY242999.1, AY242998.1, AY242997.1, AF513852.1, AF513851.1, AF063497.1 and AF028704.1, wherein, preferably the amino acid sequence is encoded by a nucleotide sequence having at least 60% identity to a nucleotide sequence in the corresponding Genbank accession number.

In one embodiment, a nucleic acid construct according to the invention is a nucleic acid construct wherein the open reading frame is an open reading frame selected from the group consisting of SEQ ID NO's: 1, 2, 14, 15, 16, 17 and 18.

In one embodiment, a nucleic acid construct according to the invention is a nucleic acid construct wherein the expression cassette comprising the sequence of SEQ ID NO: 5.

In one embodiment, a nucleic acid construct according to the invention is a nucleic acid construct wherein the nucleic acid construct is an insect cell-compatible vector, preferably a baculoviral vector.

In a second aspect, the invention pertains to an insect cell comprising a nucleic acid construct according to the invention. In one embodiment, the insect cell further comprises at least one of: i) a nucleic acid construct comprising at least one expression cassette for expression of nucleotide sequence encoding parvoviral Rep proteins; and, ii) a nucleic acid construct comprising a transgene that is flanked by at least one parvoviral inverted terminal repeat sequence. In one embodiment, the insect cell of the invention is an insect cell wherein at least one of the nucleic acid construct in i) and the nucleic acid construct in ii) is comprised in a baculoviral vector. In one embodiment, the insect cell of the invention is an insect cell wherein the nucleic acid construct in i) is stably integrated in the genome of the insect cell.

In a third aspect the invention relates to a method for producing an AAV vector in an insect cell comprising the steps of: a) culturing an insect cell as defined herein above, under conditions such that the AAV vector is produced; and, b) recovery of the AAV vector. In one embodiment of the method, the recovery of the AAV vector in step b) comprises at least one of affinity-purification of the vector using an immobilised anti-AAV antibody, preferably a single chain camelid antibody or a fragment thereof, or filtration over a filter having a nominal pore size of 30-70 nm.

In a third aspect the invention relates to an AAV vector obtainable by a method according to the invention for producing an AAV vector, wherein preferably, the AAV vector is characterised in at least one of: a) the AAV vector has an in vitro potency that does not differ by more than 10% from the potency of a corresponding AAV vector produced in mammalian cells; and, b) the AAV vector has an in vivo potency that is at least a factor 1.5 higher than the potency of a corresponding AAV vector produced in mammalian cells.

In a fourth aspect the invention relates to a pharmaceutical composition comprising an AAV vector obtainable by a method according to the invention, and a pharmaceutically acceptable carrier.

In a fifth aspect the invention relates to an AAV vector obtainable by a method according to the invention, or a pharmaceutical composition comprising the AAV vector, for use in gene therapy.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” and “similarity” also means the degree of sequence relatedness between amino acid or nucleic acid sequences, as the case may be, as determined by the match between strings of such sequences. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.). Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A “parvoviral vector” is defined as a recombinantly produced parvovirus or parvoviral particle that comprises a polynucleotide to be delivered into a host cell, either in vivo, ex vivo or in vitro. An adeno-associated virus (AAV) vector is an example of a parvoviral vector. Herein, a parvoviral or AAV vector refers to the polynucleotide comprising part of the parvoviral genome, usually at least one ITR, and a transgene, which polynucleotide is preferably packaged in a parvoviral or AAV capsid.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

Any reference to nucleotide or amino acid sequences accessible in public sequence databases herein refers to the version of the sequence entry as available on the filing date of this document.

### DETAILED DESCRIPTION OF THE INVENTION

The present inventors have set out to improve the potency of insect cell-produced neurotropic AAV vectors. Neurotropic AAV vectors, such as AAV9, when produced in insect cells have thus far shown a reduced potency as compared to the corresponding AAV vector produced in conventional mammalian cell-based systems. Similar problems with insect cell produced AAV vectors of other serotypes, such as AAV5, have been successfully addressed using a CUG as suboptimal VP1 start codon in combination with an extra alanine codon inserted immediately after the start codon (WO2015/137802), or by utilising an attenuated Kozak sequence in combination with an AUG VP1 start codon to fine tune the leaky scanning of the AUG codon (Kondratov et al., 2017, supra). These approaches have however not been successful in improving the potency insect cell-produced AAV9, as demonstrated by Kondratov et al. (2017, supra) and by the present inventors herein. The present inventors have however surprisingly discovered that the use of a non-canonical ACG initiation codon for VP1 does however allow to produce neurotropic AAV vectors, such as AAV9, in insect cells with an in vivo potency that exceeds that of the corresponding vectors produced in mammalian cells.

### Nucleic Acid Construct for Expression of AAV Capsids

In a first aspect, the invention therefore provides for a nucleic acid construct for expression of AAV capsid proteins in insect cells.

In one embodiment, a nucleic acid construct of the invention thus comprises an expression cassette for expression of AAV capsid proteins in insect cells. In one embodiment, a nucleic acid construct of the invention comprises an expression cassette comprising a promoter that is active in insect cells, the promoter being operably linked to a nucleotide sequence encoding an mRNA, the mRNA comprising an open reading frame translation of which in an insect cell produces AAV VP1, VP2 and VP3 capsid proteins, wherein the open reading frame comprises: a) an ACG codon as suboptimal VP1 translation initiation codon; and, b) a nucleotide sequence that has at least 95, 97 or 100% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon.

In one embodiment, a nucleic acid construct of the invention comprises an expression cassette comprising a promoter that is active in insect cells, the promoter being operably linked to a nucleotide sequence encoding an mRNA, the mRNA comprising an open reading frame translation of which in an insect cell produces AAV VP1, VP2 and VP3 capsid proteins, wherein the open reading frame in 5′ to 3′order (comprises or) consists of: a) an ACG codon as suboptimal VP1 translation initiation codon; b) a nucleotide sequence that has at least 95, 97 or 100% sequence identity with positions 4-42 of SEQ ID NO: 1, which nucleotide sequence encodes for amino acids 2-13 of an AAV VP1 protein; and, c) a nucleotide sequence comprising the remainder of the open reading frame translation of which in an insect cell produces AAV VP1, VP2, and VP3 capsid proteins, whereby the remainder starts at the position corresponding to amino acid position 14 of the open reading frame.

It is understood herein that a nucleotide sequence that has at least 95, 97 or 100% sequence identity with positions 4-42 of SEQ ID NO: 1, respectively has at least 37, 38 or 39 nucleotides that are identical to the 39 nucleotides of positions 4-42 of SEQ ID NO: 1.

In one embodiment, a nucleic acid construct of the invention is a nucleic acid construct wherein the nucleotide sequence in b) encodes an amino acid sequence that has at least 10, 11, or 12 amino acids that are identical to the amino acid sequence of positions 2-13 of SEQ ID NO: 12.

The nucleotide sequence that encodes the mRNA comprising an open reading frame translation of which in an insect cell produces AAV VP1, VP2 and VP3 capsid proteins thus preferably encodes a mRNA that is capable of being translated into all three AAV VP1, VP2 and VP3 capsid proteins by leaky scanning of the VP1 and VP2 translation initiation codons. Nucleotide sequence encoding an mRNA comprising an open reading frame wherein at least one of the VP1 and VP2 translation initiation codons have been inactivated are thus excluded from the invention.

In one embodiment, a nucleic acid construct of the invention is a nucleic acid construct wherein the nucleotide sequence in b) comprises at least one of i) a CTT or CTA codon in positions corresponding to position 19-21 of SEQ ID NO: 1; and ii) a CCA or CCC codon in positions corresponding to position 22-24 of SEQ ID NO: 1.

In one embodiment, a nucleic acid construct of the invention is a nucleic acid construct wherein the nucleotide sequence in b) comprises at least one of i) a CTA in positions corresponding to position 19-21 of SEQ ID NO: 1; and ii) a CCC codon in positions corresponding to position 22-24 of SEQ ID NO:1.

In one embodiment, a nucleic acid construct of the invention is a nucleic acid construct wherein the nucleotide sequence in b) is a nucleotide sequence of positions 4-42 of SEQ ID NO's: 1 or 2.

The nucleotide sequence of positions 4-42 of SEQ ID NO: 1 is a sequence that is 100% identical between AAV9 isolate hu.14 (SEQ ID NO: 1) and AAV isolate rh.10 (SEQ ID NO: 14), for both of which the inventors have found that they can be produced with high yields in insect cells using a nucleic acid construct of the invention. It can therefore be reasonably expected that a nucleic acid construct of the invention can also be used to produce other AAV serotypes, isolates and synthetic cap constructs have a similar sequence at the 5′ end of their VP1 coding sequences insect cells with high yields and/or potencies.

Therefore, in one embodiment, a nucleic acid construct of the invention is a nucleic acid construct wherein the AAV VP1, VP2, and VP3 capsid proteins are capsid proteins of an AAV serotype, isolate or synthetic cap construct selected from the group consisting of: AAV-PHP.B8 (MT162432.1), AAV-PHP.B7 (MT162431.1), AAV-PHP.B6 (MT162430.1), AAV-PHP.B5 (MT162429.1), AAV-PHP.B4 (MT162428.1), AAV-PHP.C3 (MT162427.1), AAV-PHP.C2 (MT162426.1), AAV-PHP.C1 (MT162425.1), AAV-PHP.N (MT162424.1), AAV-PHP.V2 (MT162423.1), AAV-PHP.V1 (MT162422.1), rAAV-KP2 (MN428627.1), a synthetic VP1 construct encoded by the sequence with accession no MN365014.1 (MN365014.1), AAV isolate CHC1024 (MK163936.1), AAV-PHP.eB (MF187357.1), AAV-PHP.S (MF187356.1), simian AAV isolate Cg34 (KT984498.1), AAV-PHP.A (KU056476.1), AAV-PHP.B3 (KU056475.1), AAV-PHP.B2 (KU056474.1), AAV-PHP.B (KU056473.1), AAV isolate Anc127 (KT235812.1), AAV isolate Anc126 (KT235811.1), AAV isolate Anc113 (KT235810.1), AAV isolate Anc110 (KT235809.1), AAV isolate Anc84 (KT235808.1), AAV isolate Anc83 (KT235807.1), AAV isolate Anc82 (KT235806.1), AAV isolate Anc81 (KT235805.1), AAV isolate Anc80L65 (KT235804.1), AAV isolate rh32.33 (EU368926.1), AAV isolate rh.8R (EU368925.1), AAV isolate rh.64R1 (EU368924.1), AAV isolate rh.48R2 (EU368923.1), AAV isolate rh.46 (EU368922.1), AAV isolate rh.39 (EU368921.1), AAV isolate rh.37R2 (EU368920.1), AAV isolate rh.2R (EU368919.1), AAV isolate hu.48R3 (EU368918.1), AAV isolate cy.5R4 (EU368914.1), AAV isolate cy.1R1 (EU368913.1), AAV isolate AAV6R2 (EU368911.1), AAV isolate AAV6.2 (EU368910.1), AAV isolate AAV6.1 (EU368909.1), AAV VR-355 (DQ180605.1), AAV VR-195 (DQ180604.1), AAV isolate hu.6 (AY530621.1), AAV isolate hu.48 (AY530611.1), AAV isolate hu.39 (AY530601.1), AAV isolate hu.17 (AY530582.1), AAV-9 isolate hu.14 (AY530579.1), AAV rh.64 (AY530574.1), AAV isolate rh.61 (AY530572.1), AAV isolate rh.60 (AY530571.1), AAV isolate rh.57 (AY530569.1), AAV isolate rh.55 (AY530568.1), AAV isolate rh.53 (AY530566.1), AAV isolate rh.52 (AY530565.1), AAV isolate rh.50 (AY530563.1), AAV isolate rh.49 (AY530562.1), AAV isolate rh.43 (AY530560.1), AAV isolate rh.25 (AY530557.1), AAV isolate rh.1 (AY530556.1), AAV isolate AAVbb.1 (AY243023.1), AAV isolate AAVbb.2 (AY243022.1), AAV isolate AAVcy.2 (AY243020.1), AAV isolate AAVcy.3 (AY243019.1), AAV isolate AAVcy.4 (AY243018.1), AAV isolate AAVcy.6 (AY243016.1), AAV isolate AAVrh.10 (AY243015.1), AAV isolate AAVrh.12 (AY243014.1), AAV isolate AAVrh.13 (AY243013.1), AAV isolate AAVrh.16 (AY243011.1), AAV isolate AAVrh.17 (AY243010.1), AAV isolate AAVrh.18 (AY243009.1), AAV isolate AAVrh.19 (AY243008.1), AAV isolate AAVrh.22 (AY243006.1), AAV isolate AAVrh.23 (AY243005.1), AAV isolate AAVrh.24 (AY243004.1), AAV isolate AAVrh.35 (AY243000.1), AAV isolate AAVrh.36 (AY242999.1), AAV isolate AAVrh.37 (AY242998.1), AAV isolate AAVrh.8 (AY242997.1), AAV serotype 8 (AF513852.1), AAV serotype 7 (AF513851.1), AAV serotype 1 (AF063497.1) and AAV serotype 6 (AF028704.1). The nucleotide sequences coding of these AAV capsid proteins (and their amino acid sequences) are available in the Genbank database under the accession numbers given in brackets.

In one embodiment, a nucleic acid construct of the invention is a nucleic acid construct wherein the amino acid sequences of the AAV VP1, VP2, and VP3 capsid proteins are comprised in an amino acid sequence of a Genbank accession number selected from the group consisting of: MT162432.1, MT162431.1, MT162430.1, MT162429.1, MT162428.1, MT162427.1, MT162426.1, MT162425.1, MT162424.1, MT162423.1, MT162422.1, MN428627.1, MN365014.1, MK163936.1, MF187357.1, MF187356.1, KT984498.1, KU056476.1, KU056475.1, KU056474.1, KU056473.1, KT235812.1, KT235811.1, KT235810.1, KT235809.1, KT235808.1, KT235807.1, KT235806.1, KT235805.1, KT235804.1, EU368926.1, EU368925.1, EU368924.1, EU368923.1, EU368922.1, EU368921.1, EU368920.1, EU368919.1, EU368918.1, EU368914.1, EU368913.1, EU368911.1, EU368910.1, EU368909.1, DQ180605.1, DQ180604.1, AY530621.1, AY530611.1, AY530601.1, AY530582.1, AY530579.1, AY530574.1, AY530572.1, AY530571.1, AY530569.1, AY530568.1, AY530566.1, AY530565.1, AY530563.1, AY530562.1, AY530560.1, AY530557.1, AY530556.1, AY243023.1, AY243022.1, AY243020.1, AY243019.1, AY243018.1, AY243016.1, AY243015.1, AY243014.1, AY243013.1, AY243011.1, AY243010.1, AY243009.1, AY243008.1, AY243006.1, AY243005.1, AY243004.1, AY243000.1, AY242999.1, AY242998.1, AY242997.1, AF513852.1, AF513851.1, AF063497.1 and AF028704.1, and wherein preferably the amino acid sequence is encoded by a nucleotide sequence having at least 60, 65, 70, 75, 80, 85, 90, 95, 96, 97, 98, 99 or 100% identity to a nucleotide sequence in the corresponding Genbank accession number. Thereby it is understood that the coding sequence further has the features as defined herein above for the open reading frame, translation of which in an insect cell produces AAV VP1, VP2 and VP3 capsid proteins, as comprised in the nucleic acid constructs of the invention.

In one embodiment, a nucleic acid construct of the invention is a nucleic acid construct wherein the AAV VP1, VP2, and VP3 capsid proteins are capsid proteins of an AAV serotype or isolate selected from the group consisting of: AAV6, AAV7, AAV8, AAV9 and AAVrh.10, preferably AAV9 and AAVrh.10, more preferably AAV9 and most preferably AAV9hu.14. AAV VP1, VP2, and VP3 capsid proteins are herein defined to be capsid proteins of a given AAV serotype if the neutralization titer of an AAV vector comprising the AAV VP1, VP2, and VP3 capsid proteins by a rabbit polyclonal antiserum against that given AAV serotype (the homologous serum) is at least 4, 8 or 16-fold higher than the neutralization titer by a rabbit polyclonal antiserum against another AAV serotype (heterologous serum) in reciprocal titrations, preferably as described by Gao et al. (2004, J. Virol. 78: 6381-6388). Thereby it is noted that the AAV1 and AAV6 serotypes cannot be distinguished by their serology in such reciprocal titrations (see Gao et al 2004, supra).

In one embodiment, a nucleic acid construct of the invention is a nucleic acid construct wherein the open reading frame encodes an amino acid sequence that has at least 82, 83, 84, 85, 86, 88, 90, 92, 94, 96, 98, 99 or 100% sequence identity with SEQ ID NO: 12. In one embodiment, a nucleic acid construct of the invention is a nucleic acid construct wherein the open reading frame has at least 78, 79, 80, 81, 82, 84, 86, 88, 90, 92, 94, 96, 98, 99 or 100% nucleotide sequence identity with SEQ ID NO: 1. Thereby it is understood that the open reading frame further has the features as defined herein above for the open reading frame, translation of which in an insect cell produces AAV VP1, VP2 and VP3 capsid proteins, as comprised in the nucleic acid constructs of the invention.

In one embodiment, a nucleic acid construct of the invention is a nucleic acid construct wherein the open reading frame encodes an amino acid sequence that has at least 85, 86, 87, 88, 89, 90, 92, 93, 94, 96, 98, 99 or 100% sequence identity with SEQ ID NO: 13. In one embodiment, a nucleic acid construct of the invention is a nucleic acid construct wherein the open reading frame has at least 80, 81, 82, 83, 84, 85, 86, 87, 88, 89, 90, 92, 94, 96, 98, 99 or 100% nucleotide sequence identity with SEQ ID NO: 14. Thereby it is understood that the open reading frame further has the features as defined herein above for the open reading frame, translation of which in an insect cell produces AAV VP1, VP2 and VP3 capsid proteins, as comprised in the nucleic acid constructs of the invention.

In one embodiment, a nucleic acid construct of the invention is a nucleic acid construct wherein the open reading frame encodes an amino acid sequence that has at least 84, 85, 86, 87, 88, 89, 90, 92, 93, 94, 96, 98, 99 or 100% sequence identity with SEQ ID NO: 44. In one embodiment, a nucleic acid construct of the invention is a nucleic acid construct wherein the open reading frame has at least 80, 81, 82, 83, 84, 85, 86, 87, 88, 89, 90, 92, 94, 96, 98, 99 or 100% nucleotide sequence identity with SEQ ID NO: 16. Thereby it is understood that the open reading frame further has the features as defined herein above for the open reading frame, translation of which in an insect cell produces AAV VP1, VP2 and VP3 capsid proteins, as comprised in the nucleic acid constructs of the invention.

In one embodiment, a nucleic acid construct of the invention is a nucleic acid construct wherein the open reading frame is an open reading frame selected from the group consisting of SEQ ID NO's: 1, 2 and 14-18, preferably SEQ ID NO's: 1, 2 and 14, more preferably SEQ ID NO's: 1 and 2, of which SEQ ID NO: 2 is most preferred.

In one embodiment, a nucleic acid construct of the invention is a nucleic acid construct wherein the AAV VP1, VP2, and VP3 capsid proteins are not capsid proteins of an AAV serotype selected from the group consisting of: AAV2, AAV3, AAV4 and AAV5.

In one embodiment, a nucleic acid construct of the invention is a nucleic acid construct wherein the promoter that is active in insect cells is a promoter other than a baculoviral p10 promoter. Preferably, the promoter that is active in insect cells is a baculoviral polyhedron (polH) promoter. More preferably, the baculoviral polH promoter is a polH promoter of a Autographa californica nuclear polyhedrosis virus, such as the polH promoter provided in SEQ ID NO: 9. A particularly preferred polH promoter is the short polH promoter that comprises or consists of SEQ ID NO: 10. However, other promoters that are active in insect cells and that are not the baculoviral p10 promoter are known in the art and can also be applied in the nucleic acid construct of the invention, e.g. a p35 promoter, a 4xHsp27 EcRE+minimal Hsp70 promoter, a delta E1 promoter, an E1 promoter or an IE-1 promoter and further promoters described in Summers and Smith. 1986. A Manual of Methods for Baculovirus Vectors and Insect Culture Procedures, Texas Agricultural Experimental Station Bull. No. 7555, College Station, Tex.; Luckow. 1991. In Prokop et al. Cloning and Expression of Heterologous Genes in Insect Cells with Baculovirus Vectors' Recombinant DNA Technology and Applications, 97-152; King, L. A. and R. D. Possee, 1992, The baculovirus expression system, Chapman and Hall, United Kingdom; O'Reilly, D. R., L. K. Miller, V. A. Luckow, 1992, Baculovirus Expression Vectors: A Laboratory Manual, New York; W. H. Freeman and Richardson, C. D., 1995, Baculovirus Expression Protocols, Methods in Molecular Biology, volume 39; U.S. Pat. No. 4,745,051; US2003148506; and WO 03/074714.

In one embodiment, a nucleic acid construct of the invention is a nucleic acid construct wherein the ACG suboptimal VP1 translation initiation codon is comprised in a Kozak consensus sequence that surrounds the initiation codon. The Kozak consensus sequence is herein defined as GCCRCC(NNN)G (SEQ. ID NO: 11), wherein R is a purine (i.e. A or G) and wherein (NNN) stands for the suboptimal ACG initiation codon as defined herein above. Preferably, in the Kozak consensus sequence in the construct sequence of the invention, the R is a G.

In one embodiment, a nucleic acid construct of the invention is a nucleic acid construct wherein the ACG suboptimal VP1 translation initiation codon is present in a VP2 initiator context. A VP2 initiator context is herein understood to mean a number of nucleotides preceding the non-canonical translational imitation start of VP2. In one embodiment, the VP initiator context is a nine nucleotide sequence CCTGTTAAG or a nucleotide sequence substantially homologous thereto, upstream of the suboptimal ACG translation initiation codon for the AAV VP1 capsid protein, preferably immediately upstream of the suboptimal ACG translation initiation codon, i.e. immediately adjacent to the ACG suboptimal translation initiation codon at its 5′ end. A sequence with substantial identity to the nucleotide sequence CCTGTTAAG and that will help increase expression of VP1 is e.g. a sequence which has at least 60%, 70%, 80% or 90% identity, preferably 100% identity, to the nine nucleotide sequence of CCTGTTAAG.

In one embodiment, a nucleic acid construct of the invention is a nucleic acid construct wherein the ACG suboptimal VP1 translation initiation codon is not comprised in a Kozak consensus sequence that surrounds the initiation codon.

In one embodiment, a nucleic acid construct of the invention is a nucleic acid construct wherein the ACG suboptimal VP1 translation initiation codon is not comprised in a VP2 initiator context.

In one embodiment therefore, a nucleic acid construct of the invention is a nucleic acid construct wherein the ACG suboptimal VP1 translation initiation codon is directly (operably) linked to the promoter sequence, i.e. without any sequence, such as a Kozak sequence or a VP2 initiator context, present between the 3′ end of the promoter sequence and the 5′ adenosine nucleotide of the ACG suboptimal VP1 translation initiation codon. Preferably herein, the 3′ end of the insect cell or baculoviral promoter sequence is understood to be the most 3′ nucleotide of the promoter sequence that is present immediately upstream of the translation initiation codon in the native insect or baculoviral gene from which the promoter is derived. Thus, in one embodiment, a nucleic acid construct of the invention is a nucleic acid construct wherein the ACG suboptimal VP1 translation initiation codon is directly linked to the 3′ end of the promoter sequence. In one embodiment, a nucleic acid construct according to the invention is a nucleic acid construct wherein the ACG suboptimal VP1 translation initiation codon is directly linked to the 3′ end of the promoter sequence of SEQ ID NO: 9 or 10, of which SEQ ID NO: 10 is most preferred.

In one embodiment, a nucleic acid construct of the invention the expression cassette comprises the sequence of SEQ ID NO: 5.

The use of the nucleic acid constructs as defined herein above for the production of AAV vectors in insect cells results in improved yields and/or potencies of the AAV vectors thus produced, as shown in the Examples herein. The term “potency” is herein used to mean the ability of an AAV vector to drive the expression of its genetic material, in a host cell, tissue, organ or individual transduced with the AAV vector, and can thus be determined by measuring the expression level of a transgene packaged in the AAV vector.

In one embodiment, a nucleic acid construct of the invention is an insect cell-compatible vector. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the nucleic acid construct is a baculovirus-expression vector (BEV). It is well-known that baculovirus-expression vectors are particularly suitable for the transfer of nucleic acids to insect cells and methods for their use are described for example in the above-cited references.

### The Insect Cell

In a second aspect, the invention pertains to an insect cell that comprises a nucleic acid construct of the invention as defined herein above.

An insect cell of the invention can be any cell that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including (r)AAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SeIZD2109, SeUCR1, Sf9, Sf900+, Sf21, BTI-TN-5B1-4, MG-1, Tn368, HzAml, Ha2302, Hz2E5, High Five (Invitrogen, CA, USA) and expresSF+© (U.S. Pat. No. 6,103,526; Protein Sciences Corp., CT, USA). A preferred insect cell according to the invention is an insect cell for production of recombinant parvoviral vectors, more specifically recombinant AAV vectors.

Growing conditions for insect cells in culture, and production of heterologous products in insect cells in culture are well-known in the art and described e.g. in the above cited references on molecular engineering of insect cells (see also WO2007/046703).

In one embodiment, an insect cell of the invention comprises only one type of a nucleic acid encoding AAV capsid proteins. The insect cell of the invention thus preferably comprises only a nucleic acid construct of the invention as defined herein above and the insect cell comprises no further nucleic acid constructs encoding one or more AAV capsid proteins. In particular, the insect cell does not comprise separate expression cassettes for expression of i) the VP1 protein and ii) the VP2 and VP3 proteins.

In one embodiment, an insect cell of the invention further comprises at least one of: i) a nucleic acid construct comprising at least one expression cassette for expression of nucleotide sequence encoding parvoviral Rep proteins; and, ii) a nucleic acid construct comprising a transgene that is flanked by at least one parvoviral inverted terminal repeat sequence.

In one embodiment, an insect cell of the invention thus comprises a nucleic acid construct comprising at least one expression cassette for expression of parvoviral replicases or Rep proteins. Parvoviral, especially AAV, replicases are non-structural proteins encoded by the rep gene. In wild type parvoviruses the rep gene produces two overlapping messenger ribonucleic acids (mRNA) with different length, due to an internal P19 promoter. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serve as a viral replication initiator proteins and act as replicase for the viral genome (Chejanovsky and Carter, J Virol., 1990, 64:1764-1770; Hong et al., Proc Natl Acad Sci USA, 1992, 89:4673-4677; Ni., et al., J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (Smith and Kotin, J. Virol., 1998, 4874-4881; King, et al., EMBO J., 2001, 20:3282-3291). To produce AAV from the baculoviral vectors in an insect cell platform, the presence of both Rep68 and Rep40 is not prerequisite (Urabe, et al., 2002).

A nucleotide sequence encoding a parvoviral Rep protein or encoding parvoviral Rep proteins, is herein understood as a nucleotide sequence encoding at least one of the two non-structural Rep proteins, Rep 78 and Rep52, that together are required and sufficient for parvoviral vector production in insect cells. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 8 and 9) or primates (e.g., serotypes 1 and 4). Examples of nucleotide sequences encoding parvoviral Rep proteins are given in SEQ ID NO's: 19-25.

It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2. Preferably, the nucleotide sequence encodes parvovirus Rep proteins that are functionally active in the sense that they have the required activities of viral replication initiator protein, replicase of the viral genome, DNA helicase and packaging of viral DNA into empty capsids as described above, sufficient for parvoviral vector production in insect cells. In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In one embodiment, the nucleic acid construct for expression of the parvoviral Rep proteins comprises a single expression cassette for expression of at least both the parvoviral Rep78 and Rep52 proteins. In one embodiment, the single expression cassette for expression of at least both the parvoviral Rep78 and Rep52 proteins comprises a single open reading frame encoding at least both the parvoviral Rep78 and Rep52 proteins and having a suboptimal translation initiation codon for the Rep78 coding sequence, which suboptimal initiation codon effect partial exon skipping so that both at least both the parvoviral Rep78 and Rep52 proteins are translated in the insect cell, as e.g. described in U.S. Pat. No. 8,512,981, incorporated herein by reference. Suitable suboptimal translation initiation codons include e.g. ACG, CTG, TTG and GTG. In another embodiment, the single expression cassette for expression of at least both the parvoviral Rep78 and Rep52 proteins comprises in 5′ to 3′order: (i) a first promoter linked operably to a 5′ portion of a first open reading frame of a parvovirus Rep78 protein, the first open reading frame comprising a translation initiation codon, (ii) an intron comprising a second insect cell promoter, the second promoter operably linked to a 5′ portion of an at least one additional open reading frame of a parvovirus Rep52 gene, wherein the at least one additional open reading frame comprises at least one additional translation initiation codon and overlaps with the 3′ portion of the first open reading frame, e.g. described in U.S. Pat. No. 8,945,918, incorporated herein by reference.

In another embodiment, the nucleic acid construct for expression of the parvoviral Rep proteins comprises at least two separate expression cassettes, one for expression of at least a parvoviral Rep78 protein and another for expression of at least a parvoviral Rep52 protein. Preferably, in this embodiment, the parvoviral Rep78 protein and the parvoviral Rep 52 protein comprise a common amino acid sequence comprising the amino acid sequence from the second amino acid to the most C-terminal amino acid of the parvoviral Rep 52 protein, wherein the common amino acid sequences of the parvoviral Rep78 protein and the parvoviral Rep52 protein are at least 90, 91, 92, 93, 94, 95, 96, 97, 98, 99 or 100% identical, and wherein the nucleotide sequence encoding the common amino acid sequence of the parvoviral Rep78 protein and the nucleotide sequence encoding the common amino acid sequences of the parvoviral Rep52 protein are less than 90, 89, 88, 87, 86, 85, 84, 83, 82, 81, 80, 79, 78, 77, 76, 75, 74, 73, 72, 71, 70, 69, 68, 67, 66, 60% identical, such as is described in U.S. Pat. No. 8,697,417, incorporated herein by reference. In a further embodiment, the nucleotide sequence encoding the common amino acid sequence of the parvoviral Rep78 protein has an improved codon usage bias for the cell as compared to the nucleotide sequence encoding the common amino acid sequences of the parvoviral Rep52 protein. Preferably, however, the nucleotide sequence encoding the common amino acid sequence of the parvoviral Rep52 protein has an improved codon usage bias for the cell as compared to the nucleotide sequence encoding the common amino acid sequences of the parvoviral Rep78 protein. Preferably, the difference in codon adaptation index (as defined hereinabove) between the nucleotide sequences coding for the common amino acid sequences in the parvoviral Rep78 protein and the parvoviral Rep52 protein is at least 0.1, 0.2, 0.3, 0.4, 0.5, 0.6, 0.7, 0.8, 0.9 or 1.0 whereby more preferably, the CAI of the nucleotide sequence coding for the common amino acid sequence in the parvoviral Rep52 protein is at least 0.5, 0.6, 0.7, 0.8, 0.9 or 1.0.

In one embodiment, the nucleotide sequences coding for the parvoviral Rep78 protein is SEQ ID NO: 25, coding for the wt AAV Rep78 protein and the nucleotide sequences coding for the parvoviral Rep52 selected from one of SEQ ID NO's: 20-23, each of which has been modified to have a different codon usage than the wild type Rep78 coding sequence of SEQ ID NO: 25. In a preferred embodiment, the nucleotide sequence coding for the parvoviral Rep78 protein is SEQ ID NO: 25, and is used in combination SEQ ID NO: 23 as nucleotide sequence coding for the parvoviral Rep52, the latter having been modified to differ as much as possible from SEQ ID NO: 25 in codon usage.

In one embodiment, the two separate expression cassettes for resp. the Rep78 and Rep52 proteins in the insect cell are optimised to obtain a desired molar ratio of the Rep78 to Rep52 proteins in the cell. Preferably, the combination of Rep78 and Rep52 expression cassettes in the cell produces a molar ratio of Rep78 to Rep52 in the range of 1:10 to 10:1, 1:5 to 5:1, or 1:3 to 3:1 in the (insect) cell. More preferably, the combination of Rep78 and Rep52 expression cassettes produces a molar ratio of Rep78 to Rep52 that is at least 1:2, 1:3, 1:5 or 1:10. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50). A desired molar ratio of Rep78 to Rep52 can be obtained by the choice of the promoters in respectively the Rep78 and Rep52 expression cassettes as herein further described below. Alternatively or in combination, the desired molar ratio of Rep78 to Rep52 can be obtained by using means to reduce the steady state level of the at least one of parvoviral Rep 78 and 52 proteins. Thus, in one embodiment, the nucleotide sequence encoding the mRNA for the parvoviral Rep protein comprises a modification that affects a reduced steady state level of the parvoviral Rep protein. The reduced steady state condition can be achieved for example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998. When using the two separate expression cassettes for resp. the Rep78 and Rep52 proteins in the insect cell the promoter in the Rep52 cassette is preferably stronger than the promoter in the Rep78 cassette. In one embodiment, the promoters in resp. the Rep78 and Rep52 cassettes are baculoviral promoters. In one embodiment, the promoters in resp. the Rep78 and Rep52 cassettes are distinct. In one embodiment, the Rep78 promoter is a delayed early baculoviral promoter, such as the 39k promoter. In one embodiment, the Rep52 promoter is a late or very late baculovirus promoter, such as the polH, p10, p6.9 and pSel120 promoters. In one embodiment, the late or very late baculovirus promoter that is used in the Rep52 cassette is a different promoter than the promoter used in the above-defined nucleic acid constructs comprising an expression cassette for expression of the capsid proteins.

In a preferred embodiment, the nucleotide sequence encoding at least one of parvoviral Rep protein comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding a parvoviral Rep protein comprises an open reading frame that starts with a suboptimal translation initiation codon and has no initiation codons further downstream. The suboptimal initiation codon preferably affects partial exon skipping upon expression of the nucleotide sequence in an insect cell. Preferably, the suboptimal initiation codon affects partial exon skipping in an insect cell so as to produce in the insect cell a molar ratio of Rep78 to Rep52 in the range of 1:10 to 10:1, 1:5 to 5:1, or 1:3 to 3:1. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

In one embodiment, an insect cell of the invention further comprises a nucleic acid construct comprising a transgene that is flanked by at least one parvoviral inverted terminal repeat (ITR) sequence.

In the context of the invention “at least one parvoviral inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition site for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. More preferably AAV2 ITRs are used. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between. In a preferred embodiment a transgene is flanked on either side by parvoviral inverted terminal repeat nucleotide sequences.

In the embodiments of the invention, the nucleotide sequence comprising the transgene (encoding either a gene product of interest, e.g. a protein, a nucleic acid molecule or a combination thereof, as further defined herein below) that is flanked by at least one parvoviral ITR sequence preferably becomes incorporated into the genome of a recombinant parvoviral (rAAV) vector produced in the insect cell. In one embodiment, the nucleotide sequence comprising the transgene is flanked by two parvoviral (AAV) ITR nucleotide sequences and wherein the transgene is located in between the two parvoviral (AAV) ITR nucleotide sequences. In one embodiment, the nucleotide sequence encoding a gene product of interest is incorporated into the recombinant parvoviral (rAAV) vector produced in the insect cell if it is located between two regular ITRs, or is located on either side of an ITR engineered with two D regions. Thus, in a preferred embodiment, the invention provides an insect cell, wherein the nucleotide sequence comprises two AAV ITR nucleotide sequences and wherein the at least one nucleotide sequence encoding a gene product of interest is located between the two AAV ITR nucleotide sequences.

Typically, the transgene, including ITRs and promoter & polyadenylation sequences, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

AAV sequences that may be used in the present invention for the production of a recombinant AAV virion, i.e. an AAV vector, in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the VP1, VP2, and VP3 capsid proteins for use in the context of the present invention have been defined in more detail herein above.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR, Rep, or VP can be used in place of wild-type AAV ITR, Rep, or VP sequences.

In one embodiment of an insect cell of the invention, at least one of i) the nucleic acid construct comprising at least one expression cassette for expression of the parvoviral Rep proteins; and ii) the nucleic acid construct comprising the transgene flanked by at least one parvoviral ITR; is comprised in an episomal nucleic acid construct, whereby preferably, the episomal nucleic acid construct is a baculoviral vector.

In one embodiment of an insect cell of the invention, both of i) the nucleic acid construct comprising at least one expression cassette for expression of the parvoviral Rep proteins; and ii) the nucleic acid construct comprising the transgene flanked by at least one parvoviral ITR; are comprised in a single episomal nucleic acid construct, whereby preferably, the episomal nucleic acid construct is a baculoviral vector.

In one embodiment of an insect cell of the invention, i) the nucleic acid construct comprising at least one expression cassette for expression of the parvoviral Rep proteins; and ii) the nucleic acid construct comprising the transgene flanked by at least one parvoviral ITR; are each comprised in a two separate episomal nucleic acid construct, whereby preferably, the episomal nucleic acid construct is a baculoviral vector.

In one embodiment, an insect cell of the invention is an insect cell wherein i) the nucleic acid construct comprising at least one expression cassette for expression of the parvoviral Rep proteins is integrated into the genome of the insect cell. Preferably, when integrated into the insect cell's genome, the nucleic acid construct for expression of the parvoviral Rep proteins comprises at least two separate expression cassettes, one for expression of at least a parvoviral Rep78 protein and another for expression of at least a parvoviral Rep52 protein. Preferably, in this embodiment, the parvoviral Rep78 protein and the parvoviral Rep 52 protein comprise a common amino acid sequence comprising the amino acid sequence from the second amino acid to the most C-terminal amino acid of the parvoviral Rep 52 protein, wherein the common amino acid sequences of the parvoviral Rep78 protein and the parvoviral Rep52 protein are at least 90, 91, 92, 93, 94, 95, 96, 97, 98, 99 or 100% identical, and wherein the nucleotide sequence encoding the common amino acid sequence of the parvoviral Rep78 protein and the nucleotide sequence encoding the common amino acid sequences of the parvoviral Rep52 protein are less than 90, 89, 88, 87, 86, 85, 84, 83, 82, 81, 80, 79, 78, 77, 76, 75, 74, 73, 72, 71, 70, 69, 68, 67, 66, 60% identical, such as is described in U.S. Pat. No. 8,697,417, incorporated herein by reference.

In one embodiment, the two separate Rep78 and Rep52 expression cassettes are integrated in the insect cell's genome in opposite directions of transcription. Therefore, in one embodiment, the Rep78 and Rep52 expression cassettes are both integrated on the same chromosome in the insect cell. In one embodiment, the Rep78 and Rep52 expression cassettes are both integrated on the same chromosome in the insect cell within less than 0.5, 1.0, 2.0, 5.0, 10, 20, 50 or 100 kb from each other.

In one embodiment of the insect cell of the invention, the cell comprises tightly controlled inducible expression of Rep genes stably integrated in insect cell lines by providing means for reducing leaky expression under non-induced conditions while maintaining strong expression under induced conditions. Such insect cells are also referred to as iRep cells, or simply iRep and are described in more detail in co-pending application PCT/EP2021/058798, incorporated by reference herein. Thus, in this embodiment of the cell, the two separate Rep78 and Rep52 expression cassettes, e.g. as described above, are integrated in the insect cell's genome in opposite directions of transcription, whereby both expression cassettes comprise promoters that are operably linked to at least one enhancer element is dependent on a transcriptional transregulator, wherein introduction of the transcriptional transregulator into the insect cell induces transcription from the promoters in the Rep78 and Rep52 expression cassettes. In one embodiment, the promoters in the Rep78 and Rep52 expression cassettes are baculoviral promoters, the transcriptional transregulator is a baculoviral immediate-early protein (IE1) or its spice variant (IE0) and the transcriptional transregulator-dependent enhancer element is a baculoviral homologous region (hr) enhancer element, wherein preferably the baculovirus is Autographa californica multicapsid nucleopolyhedrovirus. In one embodiment, the hr enhancer element comprises at least one copy of the hr 28-mersequence of SEQ ID NO: 26 and/or at least one copy of a of a sequence of which at least 20, 21, 22, 23, 24, 25, 26, or 27 nucleotides are identical to sequence SEQ ID NO: 26 and which binds to a baculoviral IE1 protein, and wherein the hr enhancer element, when operably linked to an expression cassette comprising a reporter gene operably linked to the polH promoter, a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and, b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element. In one embodiment, the hr enhancer element is selected from the group consisting of hr1, hr2-0.9, hr3, hr4b and hr5, of which hr2-0.9, hr4b and hr5 are preferred, of which hr4b is most preferred.

### Parvoviral Vectors

The present invention relates to nucleic acid constructs for producing recombinant parvoviruses in insect cells. The parvoviruses in particular are dependoviruses such as infectious human or simian AAV, and the components thereof (e.g., a parvovirus genome) for use as vectors for introduction and/or expression of nucleic acids in mammalian cells, preferably human cells. In particular, the invention relates to means and methods that allow for the production in insect cells of such AAV vectors.

A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wildtype (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. Wild type AAV infection in mammalian cells relies for the capsid proteins production on a combination of alternate usage of two splice acceptor sites and the suboptimal utilization of an ACG initiation codon for VP2.

A “recombinant parvoviral or AAV vector” (or “rAAV vector”) herein refers to a vector comprising one or more polynucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

### The Transgene

The nucleotide sequence comprising the transgene as defined herein above may thus comprise a nucleotide sequence encoding a gene product of interest (for expression in the mammalian cell) or encoding a nucleotide sequence targeting a gene of interest (for silencing said gene of interest in a mammalian cell), and may be located such that it will be incorporated into an recombinant parvoviral (rAAV) vector replicated in the insect cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant parvoviral (rAAV) vector produced in accordance with the present invention. The nucleotide sequence may e.g. encode a protein or it may express an RNAi agent, i.e. an RNA molecule that is capable of RNA interference such as, e.g. an shRNA (short hairpinRNA) or an siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in the insect cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidaemia or amyotrophic lateral sclerosis (ALS).

The diseases that can be treated using a recombinant parvoviral (rAAV) vector produced in accordance with the present invention are not particularly limited, other than generally having a genetic cause or basis. For example, the disease that may be treated with the disclosed vectors may include, but are not limited to, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-1), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), Parkinson's disease, phenylketonuria (PKU), spinal muscular atrophy, paralysis, Wilson disease, epilepsy, Pompe disease, amyotrophic lateral sclerosis (ALS), Tay-Sachs disease, hyperoxaluria (PH-1), spinocerebellar ataxia type 1 (SCA-1), SCA-2, SCA-3, micro-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. As preferred AAV vectors of the present invention have CNS tropism, preferred diseases that can be treated using such AAV vectors produced in accordance with the present invention are diseases, preferably genetic diseases, of the central nervous system and/or (genetic) diseases that can be treated by targeting the AAV vector to the CNS.

Examples of therapeutic gene products to be expressed include antibodies, N-acetyl-alpha-glucosaminidase, (NaGLU), Treg167, Treg289, EPO, IGF, IFN, GDNF, FOXP3, Factor VIII, Factor IX, insulin, Aromatic L-Amino Acid Decarboxylase (AADC), ApoE2, Frataxin, survival motor neuron (SMN) protein, glucocerebrosidase, N-sulfoglucosamine sulfohydrolase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLBI) and/or gigaxonin (GAN). As preferred AAV vectors of the present invention have CNS tropism, preferred therapeutic gene products are those that are useful in the treatment of genetic diseases of the central nervous system.

Examples of endogenous genes the expression of which is to be inhibited and/or modified for a therapeutic effect by targeting with an RNAi agent include: superoxide dismutase 1 (SOD1), chromosome 9 open reading frame 72 (C90RF72), TAR DNA binding protein (TARDBP), ataxin-1 (ATXN1), ataxin-2 (ATXN2), ataxin-3 (ATXN3), huntingtin (HTT), amyloid precursor protein (APP), apolipoprotein E (ApoE), microtubule-associated protein tau (MAPT), alpha-synuclein (SNCA), voltagegated sodium channel alpha subunit 9 (SCN9A), and/or voltage-gated sodium channel alpha subunit 10 (SCN10A). As preferred AAV vectors of the present invention have CNS tropism, preferred endogenous genes the expression of which is to be inhibited and/or modified for a therapeutic effect by targeting with an RNAi agent include those endogenous genes that play a role in genetic diseases of the central nervous system.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest. Many such promoters are known in the art (see Sambrook and Russel, 2001, supra). Constitutive promoters that are broadly expressed in many cell-types, such as the CMV, CAG and PGK promoters, may be used. However, more preferred will be promoters that are inducible, tissue-specific, cell-type-specific, or cell cycle-specific. For example, for liver-specific expression (as disclosed in PCT/EP2019/081743) a promoter may be selected from an α1-anti-trypsin promoter, a thyroid hormone-binding globulin promoter, an albumin promoter, LPS (thyroxine-binding globin) promoter, HCR-ApoCll hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10). For example, for neuron-specific expression a promoter may be selected from a neuron-specific enolase (NSE) promoter, platelet-derived growth factor (PDGF) promoter, platelet-derived growth factor B-chain (PDGF-(3) promoter, synapsin or synapsin-1 (Syn or Syn-1) promoter, methyl-CpG binding protein 2 (MeCP2) promoter, Ca^{+/}calmodulin-dependent protein kinase II (CaMKII) promoter, metabotropic glutamate receptor 2 (mGluR2) promoter, neurofilament light (NFL) or heavy (NFH) promoter, β-globin minigene nP2 promoter, preproenkephalin (PPE) promoter, enkephalin (Enk) promoter and excitatory amino acid transporter 2 (EAAT2) promoter. For astrocyte-specific expression a promoter may be selected from glial fibrillary acidic protein (GFAP) and EAAT2 promoters. For oligodendrocyte-specific expression the myelin basic protein (MBP) promoter a promoter may be selected. A particularly preferred promoter for expression of transgene in the peripheral and/or central nervous system is the CBh promoter (Gray et al., 2011, Hum. Gene Ther. 22:1143-1153).

In one embodiment, in case transgene/therapeutic gene product is or includes a (small) RNA molecule, such as an siRNA, shRNA, miRNA, crRNA or a guide RNA), the promoter is an RNA polymerase Ill promoter such as a promoter from a U6 snRNA gene, preferably a primate or human U6 promoter.

Various modifications of the nucleotide sequences as defined above, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

### Method for Producing a Parvoviral Vector

In a further aspect, the invention provides for a method for producing a recombinant parvoviral virion, e.g. an AAV vector. The method preferably comprises the steps of: a) culturing an insect cell as defined herein; b) providing the insect cell cultured in a) with the nucleic acid constructs as defined herein; and, c) recovery of the parvoviral vector. In one embodiment, the cell culture in a) is transfected, also known as infected, with the nucleic acid constructs as defined herein.

Recovery preferably comprises the step of affinity-purification of the recombinant parvoviral vector (i.e. the virions comprising the transgene) using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV9 capsid but at the same time also it may also specifically bind to one or more of AAV10rh, AAV8 and AAV5 capsids.

In a further embodiment, wherein recovery of the parvoviral vector in step c) comprises at least one of affinity-purification of the virion using an immobilised anti-parvoviral antibody, preferably a single chain camelid antibody or a fragment thereof, and filtration over a filter having a nominal pore size of 30-70 nm.

Therefore, in one embodiment the invention provides a method for producing a parvoviral, e.g. AAV, vector in a cell. The method preferably comprising the steps of: a) culturing an insect cell as defined herein; b) infecting the cell cultured in a) with the nucleic acid constructs as defined herein; and, c) recovery of the parvoviral vector wherein recovery of the parvoviral vector in step b) comprises at least one of affinity-purification of the vector virion using an immobilised anti-parvoviral antibody, preferably a single chain camelid antibody or a fragment thereof, or filtration over a filter having a nominal pore size of 30-70 nm.

### An AAV Vector Obtainable by a Method of the Invention and Pharmaceutical Compositions Comprising Such AAV Vectors

In another aspect, the invention pertains to an AAV vector that is obtained or obtainable in a method according to the invention. An AAV vector that is obtained or obtainable in a method according to the invention is preferably characterised in that the AAV vector has a potency that is comparable to or preferably higher than the potency of a corresponding AAV vector produced in mammalian cells, such as e.g. HEK 293 or HEK293T cells.

Thus, in one embodiment, an AAV vector obtainable by a process according the invention has an in vitro potency that does not differ by more than 20, 15, 12.5, 10, 8 or 5% from the potency of a corresponding AAV vector produced in mammalian cells. Preferably the in vitro potency of the AAV vector obtainable by a process according the invention is at least 5, 8, 10, 12.5, 15, or 20% higher than the potency of a corresponding AAV vector produced in mammalian cells. Thereby the in vitro potency of an AAV vector is preferably determined as outlined in the Examples herein. Thus, for example the AAV vector is used to transduce (or infect) a suitable in vitro cultured cell line (e.g. Huh7) at a non-saturating multiplicity of infection (MOI) and the maximum level of expression of the transgene (carried by the AAV vector) is determined at a suitable time (or time points) after infection. The maximum level of expression of the transgene is then taken as a measure of the vector's in vitro potency.

In another one embodiment, an AAV vector obtainable by a process according the invention has an in vivo potency that is at least a factor 15, 12.5, 10, 7.5, 5, 2, 1.5, 1.2 or 1.1 higher than the potency of a corresponding AAV vector produced in mammalian cells. Thereby the in vivo potency of an AAV vector is preferably determined as outlined in the Examples herein. Thus, for example the AAV vector is administered (intravenously or intrathecally) to a suitable test animal (e.g. mice, rats or non-human primates), preferably at a non-saturating dosage, and the maximum level of expression of the transgene (carried by the AAV vector) is determined at a suitable time (or time points) after administration in the relevant organs, tissues or cells, e.g. serum, cortex, liver and/or various regions of the brain. The maximum level of expression of the transgene is then taken as a measure of the vector's in vivo potency.

In a further aspect the invention relates to a batch of parvoviral vectors produced in the above described methods of the invention. A “batch of parvoviral vectors” is herein defined as all parvoviral vector virions that are produced in the same round of production, optionally per container of insect cells.

In yet a further aspect, the invention relates to a pharmaceutical composition comprising parvoviral virions, e.g. AAV vectors, produced in the above described methods of the invention, and at least one pharmaceutically acceptable carrier. Pharmaceutically acceptable carriers include, for example, water, saline, dextrose, glycerol, sucrose, orthe like, and combinations thereof. In addition, the composition may contain auxiliary substances, such as, wetting or emulsifying agents, pH buffering agents, stabilizing agents, or other reagents that enhance the effectiveness of the rAAV pharmaceutical composition.

In a again a further aspect, the invention relates to an AAV vector obtainable by a process according the invention, or a pharmaceutical composition comprising the vector, for use (as a medicament) in gene therapy.

In one embodiment, the invention relates to a method of gene therapy comprising the step of administering an effective amount of an AAV vector obtainable by a process according the invention, or a pharmaceutical composition comprising the vector, to a subject in need of gene therapy.

In one embodiment, the gene therapy is for the treatment of a disease defined herein above, preferably using a therapeutic gene as herein indicated above or using an RNAi agent for inhibiting or modifying the expression of an endogenous gene as indicated herein above.

### Kits

In a further aspect, the invention provides for a kit of parts comprising at least a nucleic acid construct as defined herein and/or further nucleic acid construct as defined herein, e.g. in the form of a baculoviral vector, for producing an AAV vector according to the invention in an insect cell. The kit can further comprises an insect cell as defined herein that can be transformed with the nucleic acid constructs.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. Stoichiometry of purified AAV9 viral capsid proteins, VP1, VP2 and VP3 as produced by the rAAV9 variants 921, 923, 919, 922 and 924 in comparison with Hek293t-produced AAV9, as analysed by electrophoretic separation (SDS-PAGE) of purified vectors.

FIG. 2. The in vitro potency of the AAV9 vector variants of Table 1.2.1. Purified AAV9 vector variants comprising the SEAP transgene were used to infect Huh7 cells at three different MOIs as indicated. 48 hours after infection activity of the transfected SEAP reporter gene was assayed.

FIG. 3. The in vitro potency of the AAV9 vector variants 921 and 924 in a side-by-side comparison with the corresponding vector produced in mammalian Hek293 cells. The effect of affinity batch binding (BB) or iodixanol (iod) purification on potency was also investigated. Purified AAV9 vector variants comprising the SEAP transgene were used to infect Huh7 cells at an MOI of 1×10⁵. 48 hours after infection activity of the transfected SEAP reporter gene was assayed.

FIG. 4. A comparison of the in vivo transduction efficiencies of the insect cell-produced AAV9 vector variant 921 comprising a human N-acetyl-alpha-glucosaminidase (NaGlu) transgene driven by the CMV promoter, with its counterpart produced in mammalian Hek293t cells. Groups of each 6 Wistar rats received an intravenous dose of 1×10¹³ genome copies of the insect cell-produced vector (AAV9 Bev), 1×10¹³ genome copies of the mammalian cell-produced vector (AAV9 Hek), or vehicle without vector. 8 weeks after administration the number of transgene copies (per μg tissue) in heart, spleen, kidney, muscle and liver was determined by Q-PCR.

FIG. 5. A comparison of the in vivo potency of the insect cell-produced AAV9 vector variant 921 comprising a human N-acetyl-alpha-glucosaminidase (NaGlu) transgene driven by the CMV promoter, with its counterpart mammalian produced in mammalian Hek293t cells. Groups of each 6 Wistar rats received an intravenous dose of 1×10¹³ genome copies of the insect cell-produced vector (AAV9 Bev), 1×10¹³ genome copies of the mammalian cell-produced vector (AAV9 Hek), or vehicle without vector. N-acetyl-alpha-glucosaminidase enzymatic activity in plasma at days −2, 8, 29 and 57 relative to the intravenous administration.

FIG. 6. A comparison of the in vivo potencies of the insect cell-produced AAV9 vector variant 921 comprising a human N-acetyl-alpha-glucosaminidase (NaGlu) transgene driven by the CMV promoter, with its counterpart mammalian produced in mammalian Hek293t cells in cerebral cortex upon intrathecal administration. Groups of each 6 Wistar rats received an intrathecal dose of 5×10¹² genome copies of the insect cell-produced vector (AAV9 Bev), 5×10¹² genome copies of the mammalian cell-produced vector (AAV9 Hek), or vehicle without vector. 8 weeks after administration the cerebral cortex was analysed for N-acetyl-alpha-glucosaminidase enzymatic activity (pmol/100 μg tissue).

FIG. 7. Graphical representation of the total pathologists counts for NaGlu-staining in the various parts of the brains as presented in Table 2.2, which summarises the microscopic findings for NaGlu staining in the histopathological examination of the brains of animals euthanized 57 days after intravenous administration or intrathecal administration (three different doses) of, respectively, the mammalian cell-produced AA9-Hek, the insect cell-produced AAV9-Bev vector, or vehicle without vector. See Table 2.2 for details.

FIG. 8. VP1, VP2 and VP3 Stoichiometry of AAV.RH10 capsid variants 1580, 1581, 1583, 1625 and 1626, as analysed by SDS-PAGE gel electrophoresis. The two clone designations refer to two baculovirus clones that were generated per AAV.RH10 variant.

FIG. 9. The in vitro potency of the AAV.RH10 vector variants of Table 2.2.1. Purified AAV.RH10 vector variants comprising the SEAP transgene were used to infect Huh7 cells at three different MOIs as indicated. 48 hours after infection activity of the transfected SEAP reporter gene was assayed.

FIG. 10. The in vitro potency of the AAV.RH10 vector variants of Table 2.2.1. Purified AAV.RH10 vector variants comprising the SEAP transgene were used to infect SH-SY5Y cells at three different MOIs as indicated. 48 hours after infection activity of the transfected SEAP reporter gene was assayed.

FIG. 11. The in vitro potency of the AAV6 vector variants of Table 3.2.1. Purified AAV6 vector variants comprising the SEAP transgene were used to infect Huh7 cells at three different MOIs as indicated. 48 hours after infection activity of the transfected SEAP reporter gene was assayed.

FIG. 12. The in vitro potency of the AAV6 vector variants of Table 3.2.1. Purified AAV6 vector variants comprising the SEAP transgene were used to infect SH-SY5Y cells at three different MOIs as indicated. 48 hours after infection activity of the transfected SEAP reporter gene was assayed.

### EXAMPLE I

### 1.1 Methods

### 1.1.1 Generation of AAV Vectors

AAV batches were generated by co-infecting expresSF+® insect cell line (Protein Sciences Corporation) with three different baculoviruses, which comprised expression cassettes for the capsid (e.g. AAV9 variants as described in Table 1.2.1), replicase and transgene. For in vitro studies the secreted embryonic alkaline phosphatase (Seap) under the control of a CMV promoter was used as reporter transgene (SEQ ID NO: 27). For in vivo studies the human N-acetylglucosaminidase, alpha (NaGlu) under the control of a PGK promoter was used as reporter transgene (SEQ ID NO: 28). Capsid expression cassettes were under the control of a polyhedron promoter. Rep expression cassettes were as described in WO 2009/14445 (BAC.VD183, SEQ ID NO:29) and under control of a deltaE1 and polyhedron promoter driving expression of Rep78 and Rep52, respectively. ExpresSF+® cells were infected at a 1:1:1 (Rep:Cap:Transgene) volumetric ratio using freshly amplified baculovirus stocks. After a 72 hour incubation at 28° C., cells were lysed with 10× lysis buffer (1,5M NaCl, 0,5M Tris-HCl, 1 mM MgC₂, 10% Triton X-100, pH=8.5) for 1 hour at 28° C., Genomic DNA was digested by Benzonase treatment for 1 hour at 37° C. Cell debris was removed by centrifugation for 15 minutes at 1900×g after which the supernatant containing the rAAV9 particles was stored at 4° C.

AAV9 material generated in Hek293t cells was produced with a two plasmid based packaging system obtained from DKFZ, Heidelberg. A plasmid comprising AAV9 hu14 Cap, AAV2 Rep expression cassettes and Adenovirus 5 helper sequences was co-transfected with a transgene plasmid comprising either a CMV-Seap or PGK-Naglu expression cassette into Hek293t cells. The AAV productions were performed at 37° C. in a 5% CO₂ humidified incubator. 72 hours after the transfection, cells were lysed for 1 hour by adding 10%, 10× lysisbuffer (1.5M NaCl, 0,5M Tris-HCl, 1 mM MgC₂, 10% Triton x-100, pH=8.5) to the medium (DMEM+10% FBS). Next, genomic DNA was digested for 1 hour at 37° C. Cell lysate was clarified by centrifugation at 1900 g for 15 minutes, after which cell supernatant was stored at 4° C. until purification. Vector titers were determined in this so-called crude cell lysate with a specific Q-PCR directed against the promoter region of the transgene (CMV-Seap) or coding sequence of the transgene (PGK-NaGlu). Virus titers of crude lysates were analysed by Q-PCR. AAVs were treated with DNAse at 37° C. to degrade extrageneous DNA. AAV DNA was then released from the particles by 1M NaOH treatment. Following a short heat treatment (30 minutes at 37° C.) the alkaline environment was neutralized with an equal volume of 1M HCl. The neutralized samples contained the AAV DNA that was used in the Taqman Q-PCR.

### 1.1.2 Purification of AAV Vectors for In Vitro Studies

AAV particles used for in vitro studies were purified from crude lysates by a batch binding protocol using Poros 9 (affinity resin, ThermoFisher). AAV crude cell lysates were added to washed (with 0.2M HPO₄ pH=7.5 buffer) resin. Subsequently, samples were incubated for 2 hours at room temperature under gentle mixing. Following the incubation the resin was washed in 0.2M HPO₄ pH=7.5 buffer and bound vectors were eluted by the addition of 0.2M Glycine pH=2.5. The pH of the eluted vectors was immediately neutralized by the addition of 0.5M Tris-HCl pH=8.5. Purified rAAV5 batches were stored at −20° C. Purified vectors were titered by a specific Q-PCR. In order to generate higher vector amounts for in vivo study a modified purification protocol was used. Briefly, following the harvest, the clarified lysate was passed over a 0.22 μm filter (Millipak 60, 0.22 μm). Next, vector particles were affinity purified by means of a 10 ml Poros 9 column (ThermoFisher) on a AKTA Avant (FPLC chromatography system, GE healthcare). Bound AAV particles were eluted from the column with 0.2M Glycine pH=2.5. The eluate was immediately neutralized by 60 mM Tris HCl pH=7.5. The buffer of the neutralized eluates was exchanged to 20 mM Tris (pH 8.0), 1 mM magnesium chloride (MgCl₂), 200 mM sodium chloride (NaCl) and 0.005% poloxamer 188 with the help of 100 KDa ultrafiltration (Millipore) filter. The final product was then filtered on a 0.22 μm filter (Millex GP), aliquoted and stored at −20° C. until further use. Following the purification virus titers were determined with a specific Q-PCR.

### 1.1.3 VP Protein Composition of AAV9 Variants

VP protein composition of purified rAAV9 variants was determined on stain free polyacrylamide gels (Biorad) stained by tryptophan staining. Briefly, 15 μl of purified rAAV9 was mixed with 5 μl 4× Laemmli loading buffer supplemented with B-mercaptoethanol (Biorad) and loaded on a Stain free polyacrylamide gel. The samples were electrophoretically separated for 35 minutes at 200 Volts. Following electrophoresis stain was developed under UV-light for 5 minutes VP proteins were then visualized under UV light on an ImageQuant system (GE Healthcare).

### 1.1.4 In Vitro Potency

To investigate in vitro potency of the different serotype 9 capsid variants, one continuous cell lines was used. 1×10⁵ Huh7 were infected with AAV9 variants as indicated at various multiplicity of infection. The experiments were performed in a 24-well plate with approximately 80% confluency at 1e5 cells/well. In both experiments wild type adenovirus was used at a multiplicity of infection of 30. This addition of wild type adenovirus is only applied in in vitro potency tests, in order to accelerate the process of second strand synthesis to within about 24 hours, thereby allowing the assay to be performed in a relatively shorter period of time and avoiding the need of cell passages. 48 hours after the start of the infection Seap expression was measured in the supernatant using the Seap reporter assay kit (Roche). Luminescence was measured on a Glowmax multi detection system (Promega) at 470 nm with an integration time of 1 second.

### 1.1.5 In Vivo Potency and Transduction Efficiency

For investigation of the in vivo potency an insect cell-produced 921 variant of AAV9 comprising the NaGlu-myc transgene (AAV9-Bev) was compared with a corresponding AAV9 vector produced in mammalian Hek293t cells (AAV9-Hek). The distribution and transgene expression of the insect cell-produced and mammalian cell produced AAV9 vectors was determined upon a single intravenous or intrathecal injection in freely moving adult male Wistar rats following an 8-week observation period. Experimental design was according to Table 1.1.1, with 6 animals per group (N=6).

TABLE 1.1.1

| | AAV | Concentration | Volume | Dose | |
|---|---|---|---|---|---|
| Group | Vector | gc/ml^{b} | (μL) | gc/animal^{b} | Route^{a} |
| 1 | 0 | 0 | 100 | 0 | IT |
| 2 | AAV9-Hek | 1 × 10¹³ | 100 | 1 × 10¹² | IT |
| 3 | AAV9-Hek | 2.5 × 10¹³ | 100 | 2.5 × 10¹² | IT |
| 4 | AAV9-Hek | 5 × 10¹³ | 100 | 5 × 10¹² | IT |
| 5 | AAV9-Bev | 1 × 10¹³ | 100 | 1 × 10¹² | IT |
| 6 | AAV9-Bev | 2.5 × 10¹³ | 100 | 2.5 × 10¹² | IT |
| 7 | AAV9-Bev | 5 × 10¹³ | 100 | 5 × 10¹² | IT |
| 8 | AAV9-Hek | 2 × 10¹³ | 500 | 1 × 10¹³ | IV |
| 9 | AAV9-Bev | 2 × 10¹³ | 500 | 1 × 10¹³ | IV |

^{a}IT = intrathecal; IV = intravenous.
^{b}gc = genome copies.

Vehicle for administration, with or without AAV, was 20 mM Tris (pH 8.0), 1 mM magnesium chloride (MgCl₂), 200 mM sodium chloride (NaCl) and 0.005% poloxamer 188.

NaGlu activity in plasma of IV-administered animals was determined at days −2, 8, 29 and 57. All surviving animals were submitted for necropsy on Day 57 (Terminal Euthanasia). There were no unscheduled deaths during the course of the study.

For the IV-administered animals genome copies/μg tissue were determined in heart, spleen, kidney, muscle and liver using a specific Q-PCR directed against the coding region of the Naglu transgene.

Histology was only performed on brain sections, Naglu was detected with a Naglu specific antibody, in addition a H&E stain was performed. Microscopic evaluation was conducted by a board-certified veterinary pathologist on the brains from all animals in all groups.

All tissues were fixed for 24 to 48 hours in paraformaldehyde before being transferred to 70% ethanol. The brains were sectioned at four levels and all sections were stained with anti-NaGlu and anti-Myc antibodies using a chromagen based detection system. NaGlu and Myc expression was detected in the cytoplasm of neurons.

The brains were sectioned at four levels and all sections were stained with anti-NaGlu using a chromagen based detection system. NaGlu expression was detected in the cytoplasm of neurons. Background staining in anti-NaGlu stained slides varied from absent to mild and in anti-NaGlu stained sections only cells with strong staining were assessed as positive. The numbers and distribution of positive staining cells was considered and the following scores assigned: 0—no staining, 1—rare/minimal scattered positive neurons, 2—mild/low numbers of scattered positive cells, 3—moderate numbers of positive cells in one or more clusters, 4—marked/large numbers of positive cells in multiple clusters, 5—severe/very large numbers of positive cells with a locally extensive distribution. Brain levels did not always contain the same structures thus to facilitate as complete a comparison as possible between animals and groups target expression was localised to major regions rather than specific nuclei or tracts. Expression in the isocortex, a substructure of the cerebral cortex, was sometimes observed in levels 1, 2 and 3. Findings recorded in the isocortex were included in the cerebral cortex values in the summary data (see Table 2.2).

### 1.2 Results

1.2.1 Generation of AAV9 Vector Variants in BEVS In order to allow for AAV9 virion production in insect cells with an improved infectivity and potency, in the present invention a series of genetic alterations in the promoter and translation initiation region of AAV9 cap expression cassettes were made as depicted in Table 2.1.

TABLE 1.2.1
Description of AAV9 capsid variants. A number of different mutations
surrounding the translational start of AAV9 VP1 were generated to
improve the stoichiometry of three VPs expressed in insect cells.
Nucleotides and amino residues changed as compared to the wild type
serotype 9 capsid sequences are underlined and in bold.

| Bac. | | VP1 | Extra | | SEQ |
|---|---|---|---|---|---|
| VD | | start | amino | Continued 5′ part of AAV9 | ID |
| No. | P_{polH}^{a} | codon | acid | VP1 capsid sequence | No. |
| 919 | 9 | ACG | — | GCTGCCGATGGTTATCTTCCAGATTGGCTCGAGGACAAC | 3 |
| | | | | A A D G Y L P D W L E D N | |
| 920 | 9 | CTG | — | GCTGCCGATGGTTATCTTCCAGATTGGCTCGAGGACAAC | 4 |
| | | | | A A D G Y L P D W L E D N | |
| 921 | 10 | ACG | — | GCTGCCGATGGTTATCTTCCAGATTGGCTCGAGGACAAC | 5 |
| | | | | A A D G Y L P D W L E D N | |
| 922 | 10 | CTG | — | GCTGCCGATGGTTATCTTCCAGATTGGCTCGAGGACAAC | 6 |
| | | | | A A D G Y L P D W L E D N | |
| 923 | 10 | ACG | GCT | GCTGCCGATGGTTATCTTCCAGATTGGCTCGAGGACAAC | 7 |
| | | | A | A A D G Y L P D W L E D N | |
| 924 | 10 | CTG | GCT | GCTGCCGATGGTTATCTTCCAGATTGGCTCGAGGACAAC | 8 |
| | | | A | A A D G Y L P D W L E D N | |

^{a}polH promoter of SEQ ID No: 9 or 10.

Baculovirus constructs harbouring all variants of AAV9 cap expression cassettes listed in Table 1.2.1 were successfully generated. Subsequently, these baculovirus cap constructs were used for generation of AAV vectors in combination with baculoviruses harbouring Rep(s) and a transgene construct, i.e. Seap as reporter gene flanked by ITRs. One of the tested constructs, i.e. VD920, irrespectively of multiple attempts did not support generation of AAV vector production. All the other constructs listed in Table 1.2.1 resulted in successful generation of AAV.

The three viral proteins (VPs) of successfully produced rAAV9 variants were isolated. The stoichiometry of the three VPs was investigated by electrophoretic separation (SDS-PAGE) of purified vectors (FIG. 1) and compared with Hek293t-produced AAV9. With the exception of construct VD920, all investigated AAV9 cassette designs resulted in similar stoichiometry to Hek293t produced AAV9. Expression cassette variations similar to AAV5 were introduced in the AAV9 cassettes. However in contrast to AAV5, where we found that small modifications around the VP1 start codon of the cap5 gene had profound influence on capsid protein stoichiometry, for AAV9 we found that this influence was only minor.

### 1.2.2 In Vitro Potency of AAV9 Vector Variants

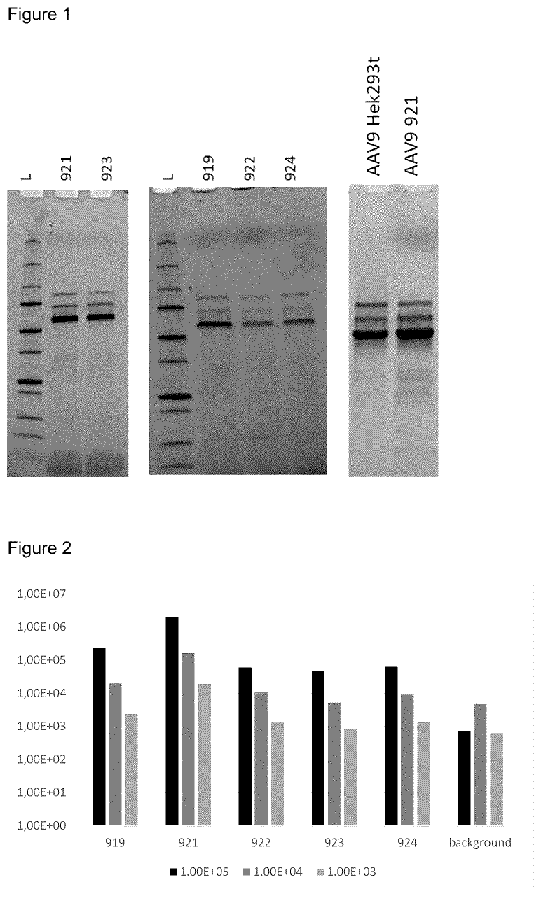

The in vitro potency of the AAV9 vector variants of Table 1.2.1 was determined using the Huh7 cell line. Purified AAV9 vector variants were used to infect Huh7 cells at three different MOIs 10 as depicted in FIG. 2. 48 hours after infection, enzymatic activity of the transfected SEAP reporter gene was assayed. Results are shown in FIG. 2. Of the five AAV9 vector variants tested, the 921 variant clearly outperforms the other variants tested by at about one order of magnitude.

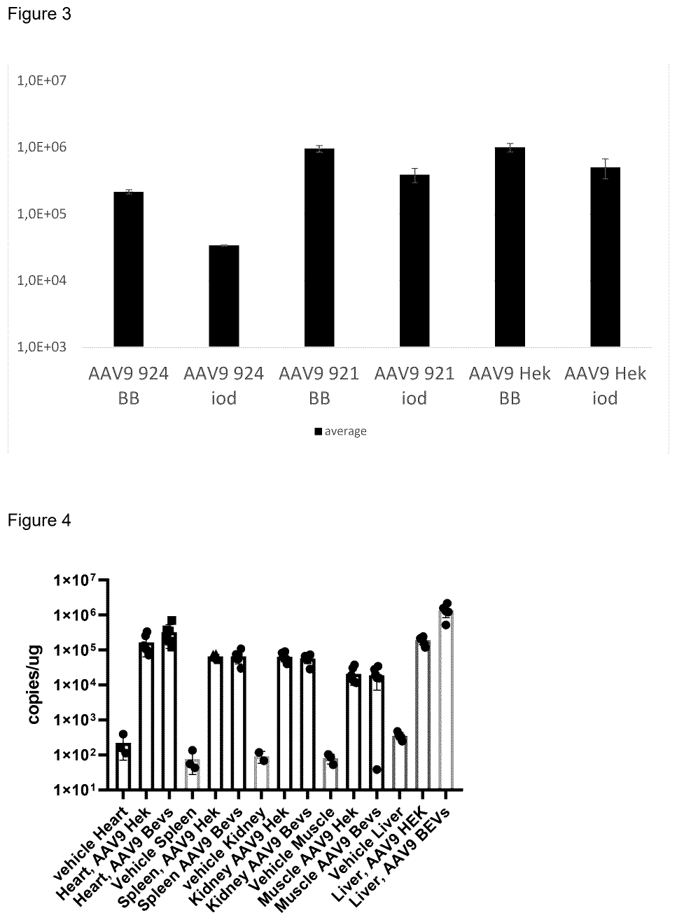

Next the in vitro potency of the AAV9 vector 921 and 924 variants was tested in a side-by-side comparison with the potency of an AAV9 vector produced in mammalian Hek293 cells. FIG. 3 shows that the in vitro potency of the 921 variant produced in insect cells is at the same level as that of the mammalian cell-produced AAV9, whereas the potency of the 924 variant is about one order of magnitude lower.

Interestingly the capsid stoichiometry of AAV9 does not appear to significantly affect potency, which is in contrast to what we previously found for AAV5. Variations in capsid cassette design which resulted in profound capsid stoichiometry shifts in AAV5 only result in minor stoichiometry changes in AAV9. Nonetheless, the variations in cassette design do still result in significant shifts in potency within our AAV9 capsid set, which is similar to what we found for AAV5.

1.2.3 In Vivo Potency and Transduction Efficiency of AAV9 Vector Variant 921 Compared with its Mammalian Cell-Produced Counterpart

Initial in vivo trials in C57BL/6 mice with an insect cell-produced 924 variant of AAV9 comprising the SEAP transgene showed that it's in vivo potency was significantly worse than the corresponding AAV9 vector produced in mammalian Hek293t (data not shown).

Next we set out to test the in vivo potency of our AAV9 variant with the highest in vitro potency, i.e. the 921 variant. To this end, the in vivo potency of an insect cell-produced 921 variant of AAV9 comprising a NaGlu-myc transgene (AAV9-Bev) was compared with a corresponding AAV9 vector produced in mammalian Hek293t cells (AAV9-Hek) after intravenous administration or intrathecal administration of the vectors to groups of adult male Wistar rats (N=6) as indicated in Table 1.1.1.

After 8 weeks the number of transgene copies (per μg tissue) in heart, spleen, kidney, muscle and liver was determined by Q-PCR in the groups that received an intravenous dose of 1×10¹³ genome copies of AAV9-Hek or AAV9-Bev, respectively. FIG. 4 shows that the in vivo transduction efficiency of the insect cell-produced AAV9-Bev vector at least equals that of the corresponding mammalian cell-produced vector. In heart and even more so in liver the insect cell-produced AAV9-Bev vector clearly has a higher transduction efficiency than the mammalian cell-produced AAV9-Hek vector.

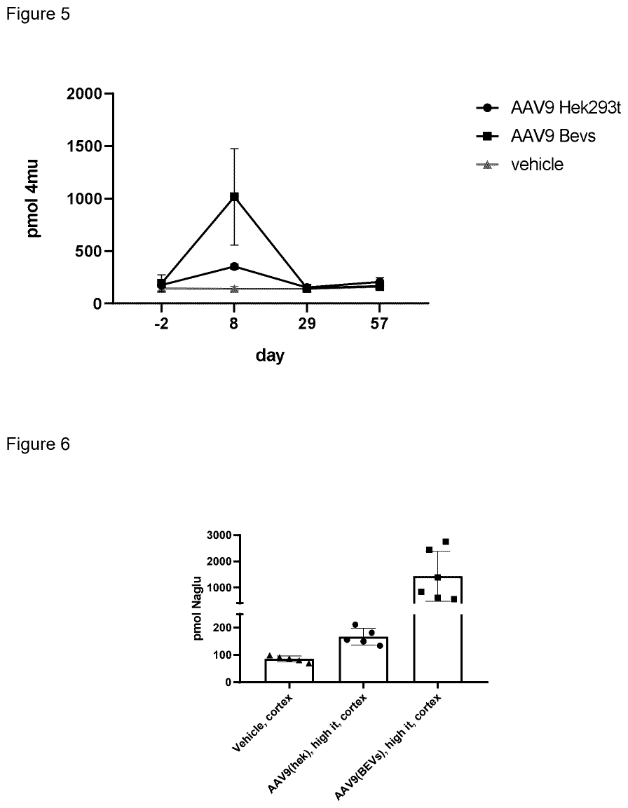

This difference is even more pronounced when looking at the plasma levels of the transgene's enzymatic NaGlu activity, which peaked 8 days post administration for both the insect cell- and mammalian cell-produced vectors but the maximum level achieved by the insect cell-produced vector was about 3 times higher than that of the mammalian cell-produced vector (see FIG. 5). The insect cell-produced vector thus clearly outperforms the mammalian cell-produced vector in terms of potency upon intravenous administration. The sharp decline of the NaGlu activity levels in plasma as seen at days 29 and 57 post administration can be explained by the rats' immune responses against cells expressing the foreign human transgene.

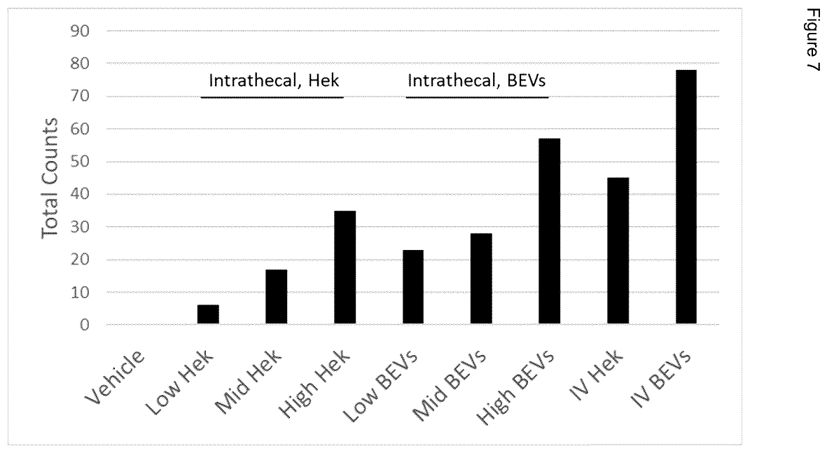

Upon intrathecal administration an even greater difference between the insect cell- and mammalian cell-produced vectors was observed in terms of level of expression of the NaGlu activity. At the highest doses the insect cell-produced AAV9-Bev vector gave an eight-fold difference in NaGlu activity in the cerebral cortex than the mammalian cell-produced AAV9-Hek (FIG. 6).

Histopathological examination confirmed that intrathecal administration of the insect cell-produced AAV9-Bev vector was associated with greater neuronal NaGlu expression than the administration of the mammalian cell-produced AA9-Hek vector (FIG. 7 and Table 1.2.2). Similar expression levels were seen with low and mid doses of the insect cell-produced vector with considerably higher expression observed at the high dose (FIG. 7 and Table 1.2.2). NaGlu expression was particularly pronounced in the cerebral cortex of levels 1 and 2 especially in the isocortex. Expression was also consistently seen in neurons in the medulla and midbrain of the brain stem and in the Purkinje cells of the cerebellum (Table 1.2.2).

Intravenous administration of the insect cell-produced AAV9-Bev vector was also associated with higher expression of NaGlu in the brain than the intravenous administration of the mammalian cell-produced AAV9-Hek vector (FIG. 7 and Table 1.2.2). Expression of NaGlu was also observed more widely with intravenous administration than with in intrathecal administration, particularly for the insect cell-produced AAV9-Bev vector (Table 1.2.2).

In conclusion, the expression of the NaGlu transgene in rat brains was significantly higher with both intrathecal and intravenous administration of the insect cell-produced AAV9-Bev vector than the mammalian cell-produced AA9-Hek vector. Intravenous administration of mammalian cell- and insect cell-produced vectors were both associated with higher and more widespread expression of the NaGlu transgene than their intrathecal administered counterparts.

TABLE 1.2.2
Summary of microscopic findings for NaGlu staining in the histopathological examination of the brains of euthanized animals
at day 57 after administration of, respectively, the mammalian cell-produced AA9-Hek, the insect cell-produced AAV9-Bev
vector, or vehicle without vector. Pathologists counts for NaGlu-staining in various parts of the brains are indicated.

| | Compound | | | | | | | | |
|---|---|---|---|---|---|---|---|---|---|
| | Vehicle | AA9-Hek | | | AAV9-Bev | | | AA9-Hek | AAV9-Bev |
| | Dose^{a} | | | | | | | | |
| | 0 | 1 × 10¹² | 2.5 × 10¹² | 5 × 10¹² | 1 × 10¹² | 2.5 × 10¹² | 5 × 10¹² | 1 × 10¹³ | 1 × 10¹³ |
| | Route | | | | | | | | |
| | Intrathecal | | | | | | | Intravenous | |
| | Group | | | | | | | | |
| | 1 | 2 | 3 | 4 | 5 | 6 | 7 | 8 | 9 |
| | No. animals | | | | | | | | |
| | 6 | 6 | 6 | 6 | 6 | 6 | 6 | 6 | 6 |
| Cerebral cortex (level 1) | 0 | 1 | 4 | 6 | 4 | 1 | 13 | 6 | 5 |
| Cerebral cortex (level 2) | 0 | 1 | 1 | 9 | 3 | 6 | 12 | 6 | 10 |
| Cerebral cortex (level 3) | 0 | 0 | 1 | 3 | 2 | 0 | 1 | 4 | 2 |
| Cerebral nuclei (level 1) | 0 | 0 | 2 | 1 | 4 | 1 | 0 | 5 | 6 |
| Cerebral nuclei (level 2) | 0 | 0 | 1 | 2 | 0 | 0 | 0 | 2 | 6 |
| Thalamus | 0 | 2 | 1 | 0 | 2 | 2 | 4 | 0 | 9 |
| Hippocampus | 0 | 0 | 0 | 0 | 0 | 0 | 0 | 4 | 8 |
| Hypothalamus | 0 | 0 | 0 | 0 | 1 | 0 | 4 | 0 | 2 |
| Midbrain | 0 | 1 | 1 | 6 | 1 | 4 | 6 | 3 | 6 |
| Medulla | 0 | 1 | 3 | 4 | 3 | 7 | 9 | 8 | 12 |
| Cerebellum | 0 | 0 | 3 | 4 | 3 | 7 | 8 | 7 | 12 |
| Total | 0 | 6 | 17 | 35 | 23 | 28 | 57 | 45 | 78 |

^{a}genome copies (gc) per animal

### EXAMPLE II

### 2.1. Methods

### 2.1.1 Generation of AAV.RH10 Vectors

AAV batches were generated by co-infecting expresSF+® insect cell line (Protein Sciences Corporation) with three different baculoviruses, which comprised expression cassettes for the capsid (e.g. AAV.RH10 variants as described in Table 2.2.1), replicase and transgene. For in vitro studies the secreted embryonic alkaline phosphatase (Seap) under the control of a CMV promoter was used as reporter transgene. Capsid expression cassettes were under the control of a polyhedron or P10 promoter. Rep expression cassettes were as described in WO 2009/14445 (BAC.VD183) and under control of a deltaE1 and polyhedron promoter driving expression of Rep78 and Rep52, respectively. ExpresSF+® cells were infected at a 1:1:1 (Rep:Cap:Transgene) volumetric ratio using freshly amplified baculovirus stocks. After a 72 hour incubation at 28° C., cells were lysed with 10× lysis buffer (1,5M NaCl, 0,5M Tris-HCl, 1 mM MgCl₂, 10% Triton X-100, pH=8.5) for 1 hour at 28° C., Genomic DNA was digested by Benzonase treatment for 1 hour at 37° C. Cell debris was removed by centrifugation for 15 minutes at 1900×g after which the supernatant containing the rAAV.RH10 particles was stored at 4° C.

### 2.1.2 Purification of AAV.RH10 Vectors

AAV.RH10 particles were purified from crude lysates by a batch binding protocol using AVB sepharose affinity resin (GE Healthcare). AAV crude cell lysates were added to washed (with 0.2M HPO₄ pH=7.5 buffer) resin. Subsequently, samples were incubated for 2 hours at room temperature under gentle mixing. Following the incubation the resin was washed in 0.2M HPO₄ pH=7.5 buffer and bound vectors were eluted by the addition of 0.2M Glycine pH=2.5. The pH of the eluted vectors was immediately neutralized by the addition of 0.5M Tris-HCl pH=8.5. Purified rAAV5 batches were stored at −20° C.

### 2.1.3 Titration of AAV.RH10 Vectors by Q-PCR

Vector titers of purified AAV.RH10 was determined with a specific Q-PCR directed against the promoter region of the transgene (CMV-Seap). AAVs were treated with DNAse at 37° C. to degrade extrageneous DNA. AAV DNA was then released from the particles by 1M NaOH treatment. Following a short heat treatment (30 minutes at 37° C.) the alkaline environment was neutralized with an equal volume of 1 M HCl. The neutralized samples contained the AAV DNA that was used in the Taqman Q-PCR.

### 2.1.4 VP Protein Composition of AAV.RH10 Vectors

VP protein composition of purified AAV.RH10 vectors was determined on stain free polyacrylamide gels (Biorad) which where stained by tryptophan staining. Briefly, 15 μl of purified rAAV.RH10 was mixed with 5 μl 4× Laemmli loading buffer supplemented with B-mercaptoethanol (Biorad) and loaded on a Stain free polyacrylamide gel. The samples were electrophoretically separated for 35 minutes at 200 Volts. Following electrophoresis stain was developed under UV-light for 5 minutes after which VP proteins were then visualized under UV light on a Chemidoc imaging system (Biorad).

### 2.1.5 In Vitro Potency of AAV.RH10 Variants in Huh7 and SH-SY5Y Cells

To investigate in vitro potency of the BEVS adapted AAV.RH10 capsid variants, two continuous cell lines were used (Huh7 and SH-SY5Y cells). 1×10⁵ Huh7 or SH-SY5Y cells were infected with AAV.RH10 variants at multiplicity of infections of 1×10⁶, 1×10⁵ and 1×10⁴. Wild type adenovirus was co-infected at a multiplicity of infection of 30. 48 hours after the start of the infection Seap reporter expression was measured in the supernatant using the Seap reporter assay kit (Roche). Luminescence was measured on a Glowmax multi detection system (Promega) at 470 nm with an integration time of 1 second.

### 2.2. Results

### 2.2.1 Adaptation of AAV.RH10 to the Baculovirus Expression System

Adaptation of the capsid expression cassette is essential if high titer and potent AAV vectors are to be produced in the insect cells. In this example we adapted the AAV.RH10 serotype for production with the baculovirus expression system. Nine AAV.RH10 capsid cassettes were designed with genetic alterations that focused on the promoter and VP1 translation initiation region constructs (Table 2.2.1). Expression cassette variations similar to AAV5 and AAV9 were introduced in the AAV.RH10 cassettes. These changes were aimed at optimizing the stoichiometry of viral capsid proteins 1, 2 and 3.

Baculovirus constructs for AAV.RH10 variants 1580, 1581, 1583, 1625 and 1626 were successfully generated. Next, these baculovirus cap constructs were used to produce AAV by combining them in equal volumetric ratio's with baculoviruses comprising a Rep and transgene cassette (Seap reporter gene flanked by AAV2 ITRs). AAV.RH10 variants 1580, 1581, 1625 and 1626 were able to produce AAV, while AAV.RH10 variant vd1583 failed to produce measurable AAV vector titers (Table 2.2.1). Of note is that AAV productions with the RH10 variants that used an ACG start codon resulted in higher vector titers than RH10 variants that used the CTG start codon. The AAV.RH10 variant that used the P10 promoter did not result in measurable vector titers at all.

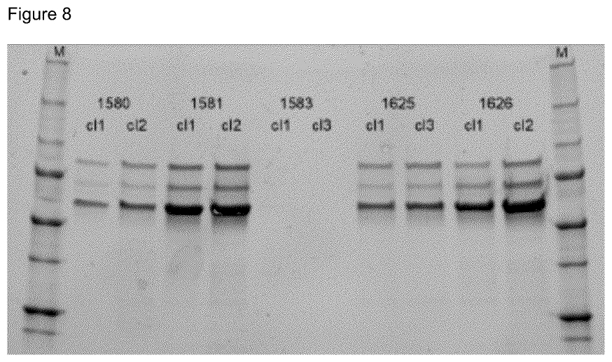

Following affinity purification of the produced AAV.RH10 vectors, the stoichiometry of the three viral proteins was investigated by SDS-PAGE gel electrophoresis (FIG. 8). With the exception of construct 1583 (which did not produce), all AAV.RH10 variants produced AAV capsids with stoichiometries that roughly fell within the expected range of 1:1:10 for VP1:VP2:VP3 respectively. AAV.RH10 that use the weaker CTG start codon have a slightly higher VP1 content in the produced AAV particles, while the amount of VP1 in the ACG start codon variants is slightly lower. The impact of small VP stoichiometry variations on vector potency needs to be carefully evaluated in in vitro and in vivo experiments.

### 2.2.2 In Vitro Potency of AAV.RH10 Variants in Huh7 and SH-SY5Y Cells

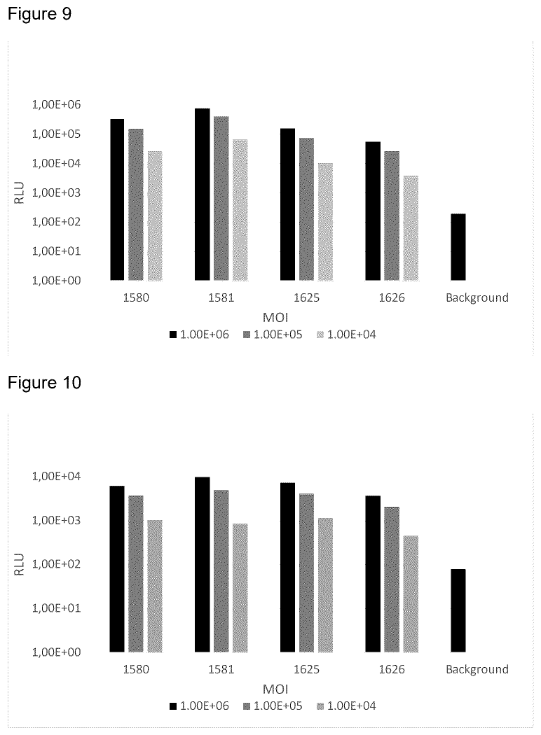

In vitro potency of BEVs adapted AAV.RH10 variants was assessed in Huh7 and SH-SY5Y cells. Both celllines were infected with batch binding purified AAV.RH10 variants at three different MOIs. Next, Seap reporter activity was measured 48 hours after the start of the infection in the cell supernatant. Variant 1581 outperformed the other variants in both Huh7 and SH-SY5Y cells (FIGS. 9 and 10, respectively). Variant 1581 follow the same design strategy as the optimal AAV9 variant and combines the polyhedrin short promoter with the ACG start codon.

### Example III

### 3.1. Methods

### 3.1.1 Generation and Purification of AAV6 Vectors

AAV batches were produced by co-infecting expresSF+ insect cells with three different baculoviruses comprising replicase, transgene and capsid cassettes. Capsid cassettes used for the experiment are described in Table 3.2.1. The secreted embryonic alkaline phosphatase (SEAP) gene was used under control of the CMV promoter as the transgene. expresSF+ insect cells were co-infected with freshly amplified baculovirus stocks at a volumetric ratio of 1:1:1 (Cap:Rep:Transgene). Following 72 hour of incubation at 28° C., cells were lysed with 10× lysis buffer (1,5M NaCl, 0,5M Tris-HCl, 1 mM MgCl₂, 10% Triton X-100, pH=8.5) for 1 hour at 28° C., Genomic DNA was digested by Benzonase treatment for 1 hour at 37° C. Cell debris was removed by centrifugation for 15 minutes at 1900×g after which the supernatant containing either the AAV6 particles was stored at 4° C. AAV6 particles were purified from crude lysates with a batch binding protocol using AVB sepharose affinity resin (GE Healthcare). AAV crude cell lysates were added to washed (with 0.2M HPO₄ pH=7.5 buffer) resin. Subsequently, samples were incubated for 2 hours at room temperature under gentle mixing. Following the binding the resin was washed in 0.2M HPO₄ pH=7.5 buffer and bound vectors were eluted by the addition of 0.2M Glycine pH=2.5. The pH of the eluted vectors was immediately neutralized by the addition of 0.5M Tris-HCl pH=8.5. Purified AAV batches were stored at −20° C.

### 3.1.2 Titration of AAV6 Vectors by QPCR

Titers of purified AAV6 batches were determined with a Q-PCR specific for the CMV promoter of the transgene (CMV-Seap). AAVs were treated with DNAse at 37° C. to degrade extrageneous DNA. AAV DNA was then released from the particles by a short (30 min) heat treatment (37° C.) in the presence of 1M NaOH. Next the alkaline environment was neutralized by the addition of an equal volume of 1M HCl. Neutralized DNA was diluted 10× in WFI 16 ng/ul PolyA, after which the samples were used in a QPCR with primers specific for the CMV promoter of the transgene.

### 3.1.3 VP Protein Composition of AAV6 Vectors

VP protein composition of purified AAV6 vectors was determined by SDS page gel electrophoresis. Briefly, 15 μl of purified AAV6 was mixed with 5 μl 4× Laemmli loading buffer supplemented with B-mercaptoethanol (Biorad). Following a short heat treatment to denature the proteins (5 min at 95° C.) material was loaded on a Stain free polyacrylamide gel (Biorad). Next, the samples were electrophoretic ally separated for 35 minutes at 200 Volts. Following electrophoresis stain (tryptophan based) was developed under UV-light for 5 minutes after which VP proteins were then visualized under UV light on a Chemidoc imaging system (Biorad).

TABLE 2.2.1
Description of AAV.RH10 variants and their producibility in insect cells.
The AAV.RH10 capsid expression cassette was adapted for production in
insect cells by swapping the promoter and mutating the translational start
site of VP1. Nucleotides and amino residues changed as compared to the wild
type serotype RH10 capsid sequences are underlined.In addition the
producibility of the RH10 variants is included as well.

| Bac. | SEQ | | VP1 | Extra | | Gc/ml |
|---|---|---|---|---|---|---|
| VD | ID | | start | amino | Continued 5′ part of AAV9 VP1 | in |
| No. | NO: | Promoter | codon | acid | capsid sequence | CLB^{b} |
| 1580 | 30 | PolH^{a} | CTG | — | GCTGCCGATGGTTATCTTCCAGATTGGCTCGAGGACAAC | 7.7e9 |
| | | | | | A A D G Y L P D W L E D N | |
| 1581 | 31 | PolH^{a} | ACG | — | GCTGCCGATGGTTATCTTCCAGATTGGCTCGAGGACAAC | 4e11 |
| | | | | | A A D G Y L P D W L E D N | |
| 1582 | 32 | P10 | ACG | — | GCTGCCGATGGTTATCTTCCAGATTGGCTCGAGGACAAC | Bac |
| | | | | | A A D G Y L P D W L E D N | failed |
| 1583 | 33 | P10 | CTG | — | GCTGCCGATGGTTATCTTCCAGATTGGCTCGAGGACAAC | Did not |
| | | | | | A A D G Y L P D W L E D N | produce |
| 1584 | 34 | P10 | ATG | — | GCTGCCGATGGTTATCTTCCAGATTGGCTCGAGGACAAC | Bac |
| | | | (double | | A A D G Y L P D W L E D N | failed |
| | | | ATG) | | | |
| 1625 | 35 | PolH^{a} | CTG | GCT | GCTGCCGATGGTTATCTTCCAGATTGGCTCGAGGACAAC | 2e10 |
| | | | | A | A A D G Y L P D W L E D N | |
| 1626 | 36 | PolH^{a} | ACG | GCT | GCTGCCGATGGTTATCTTCCAGATTGGCTCGAGGACAAC | 1.2e11 |
| | | | | A | A A D G Y L P D W L E D N | |
| 1627 | 37 | P10 | ACG | GCT | GCTGCCGATGGTTATCTTCCAGATTGGCTCGAGGACAAC | Bac |
| | | | | A | A A D G Y L P D W L E D N | failed |
| 1628 | 38 | P10 | CTG | GCT | GCTGCCGATGGTTATCTTCCAGATTGGCTCGAGGACAAC | Bac |
| | | | | A | A A D G Y L P D W L E D N | failed |

^{a}polH promoter of SEQ ID No: 10.
^{b}genome copies per ml in crude lysis buffer.

TABLE 3.2.1
Description of AAV6 variants and their producibility in insect cells.
The AAV6 capsid expression cassette was adapted for production in insect
cells by swapping the promoter and mutating the translational start site
of VP1. Nucleotides and amino residues changed as compared to the
wild type serotype AAV6 capsid sequences are underlined. In addition
the producibility of the AAV6 variants is included as well.

| Bac. | SEQ | | VP1 | AAV | | |
|---|---|---|---|---|---|---|
| VD | ID | | start | sero- | Continued 5′ part of AAV6 VP1 capsid | |
| No. | NO: | PpolH^{a} | codon | type | sequence | Gc/ml in CLB^{b} |
| 1083 | 39 | 9 | CTG | AAV6 | GCTGCCGATGGTTATCTACCCGATTGGCTCGAGGACAAC | 5.5e11 |
| | | | | | A A D G Y L P D W L E D N | |
| 1084 | 40 | 10 | CTG | AAV6 | GCTGCCGATGGTTATCTACCCGATTGGCTCGAGGACAAC | Did not produce |
| | | | | | A A D G Y L P D W L E D N | |
| 1085 | 41 | 9 | GTG | AAV6 | GCTGCCGATGGTTATCTACCCGATTGGCTCGAGGACAAC | 7.70e11 |
| | | | | | A A D G Y L P D W L E D N | |
| 1086 | 42 | 10 | GTG | AAV6 | GCTGCCGATGGTTATCTACCCGATTGGCTCGAGGACAAC | Did not produce |
| | | | | | A A D G Y L P D W L E D N | |
| 1087 | 43 | 9 | ACG | AAV6 | GCTGCCGATGGTTATCTACCCGATTGGCTCGAGGACAAC | 2.60e12 |
| | | | | | A A D G Y L P D W L E D N | |

^{a}polH promoter of SEQ ID No: 9 or 10.
^{b}genome copies per ml in crude lysis buffer.

### 3.1.4 In Vitro Potency of and AAV6 Variants in Huh7 and SH-SY5Y Cells

To investigate in vitro potency of the BEVS adapted AAV6 capsid variants, two continuous cell lines were used (Huh7 and SH-SY5Y cells). 1×10⁵ Huh7 or SH-SY5Y cells were infected with AAV6 variants at a multiplicity of infections of 1×10⁶, 1×10⁵ and 1×10⁴. For all experiments wild type adenovirus was used at a multiplicity of infection of 30. This addition of wild type adenovirus is only applied in in vitro potency tests, in order to accelerate the process of second strand synthesis to within about 24 hours, thereby allowing the assay to be performed in a relatively shorter period of time and avoiding the need of cell passages. 48 hours after the start of the infection Seap reporter expression was measured in the supernatant using the Seap reporter assay kit (Roche). Luminescence was measured on a Glowmax multi detection system (Promega) at 470 nm with an integration time of 1 second.

### 3.2 Results

### 3.2.1 Molecular Adaptation of AAV6 to the Baculovirus Expression System

Efficient production of potent rAAV in insect cells requires the adaptation of the capsid expression cassette. By optimizing the VP1 start codon area (promoter and VP1 initiator triplet) of AAV6 we aimed to balance VP1:VP2:VP3 expression ratio of these capsids towards their natural stoichiometry of 1:1:10. Two versions of the polyhedrin promotor (long and short) were combined with non-canonical start codons (CTG, GTG and ACG) to generate five versions of the AAV6 expression capsid cassette.

Baculovirus constructs were successfully generated with the five designs listed in Table 3.2.1. In the following step AAVs were produced in insect cells by combining the Cap baculoviruses in 1:1:1 volumetric ratio's with baculoviruses comprising Rep and transgene (CMV-Seap) expression cassettes. AAV6 variants 1084 and 1087 did not produce, whereas the three remaining designs did (1083, 1085 and 1087, Table 3.2.1). Following AAV production, particles were purified with AVB Sepharose using a batch binding protocol. Purified AAV batches were electrophoretically separated and VP123 proteins were visualized on SDS-PAGE gels. Similar to the adaptations of AAV rh10 and AAV9, capsid stoichiometries that roughly corresponded to wild type where observed with all the AAV6 variants that managed to produce measurable titers (data not shown).

### 3.2.2 In Vitro Potency of AAV6 Variants in Huh7 and SH-SY5Y Cells

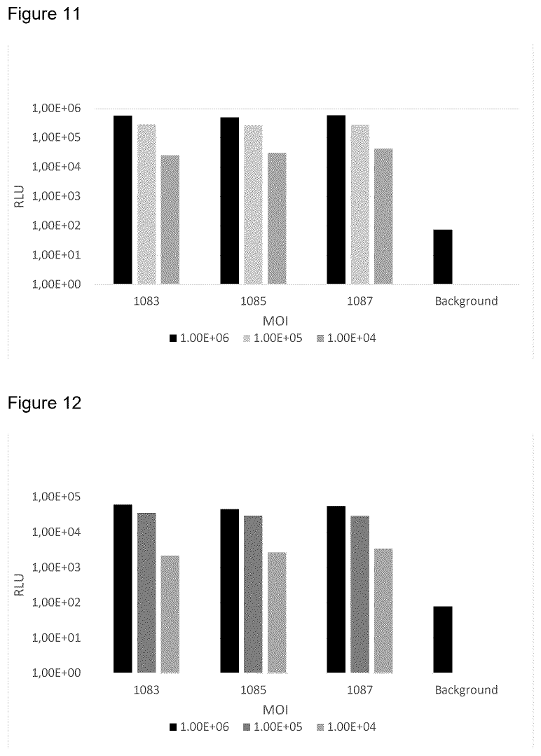

In vitro potency of BEVs adapted AAV6 variants was determined in Huh7 and SH-SY5 Ycells. Huh7 and SH-SY5Y cells were infected with three different MOIs of purified AAV6 variants. Seap reporter activity was measured 48 hours after infection in the cell supernatant. We found no major differences in in vitro potency between the variants in both Huh7 and SH-SY5Y cells (FIGS. 11 and 12, respectively).

### 3.3 Conclusion

AAV6 capsids cassettes were adapted for the production of rAAV in insect cells. Several capsid cassette designs were compared based on their productivity, capsid stoichiometry and in vitro potency. The VP1 start codon of the design had significant impact on productivity. Of note is that the highest AAV vector yields were achieved with variants that used ACG as the VP1 start codon. These results are in line with the BEVs adaptations of AAV9 and AAV rh10 (Example I and II, above), where we also found that the ACG codon yielded the highest producing variants.

Stoichiometries roughly similar to wild type (VP1:VP2:VP3 of 1:1:10) were found with all designs that managed to produce measurable vector titers. We noted that the CTG start codon resulted in slightly improved incorporation of the VP1 protein into the AAV6 capsid, while slightly reducing the vector titers. In contrast the ACG start codon resulted in slightly lower VP1 incorporation, but higher vector titers. Similar to AAV9 (and unlike AAV5), AAV6 in vitro potency did not appear to be influenced by the small observed stoichiometry changes.

1. A nucleic acid construct comprising an expression cassette comprising a baculoviral polH promoter, operably linked to a nucleotide sequence encoding an mRNA, comprising an open reading frame translation of which in an insect cell produces AAV VP1, VP2, and VP3 capsid proteins, wherein the open reading frame comprises:
a) an ACG codon as suboptimal VP1 translation initiation codon; and,
b) a nucleotide sequence that has at least 95% sequence identity with positions 4-42 of SEQ ID NO: 1, which nucleotide sequence encodes for amino acids 2-13 of an AAV VP1 protein and which nucleotide sequence immediately follows the ACG suboptimal VP1 translation initiation codon,
and wherein the open reading frame encodes an amino acid sequence that has at least 85% sequence identity with SEQ ID NO: 12.

2. A nucleic acid construct according to claim 1, wherein the promoter is a polH promoter of Autographa californica nuclear polyhedrosis virus.

3. A nucleic acid construct according to claim 2, wherein the baculoviral polH promoter comprises or consists of the nucleotide sequence in SEQ ID NO: 10.

4. A nucleic acid construct according to claim 3, wherein the ACG suboptimal VP1 translation initiation codon is directly linked to the 3′ end of the promoter sequence.

5. A nucleic acid construct according to claim 1, wherein the nucleotide sequence in b) comprises at least one of i) a CTA codon in positions corresponding to position 19-21 of SEQ ID NO: 1; and ii) a CCC codon in positions corresponding to position 22-24 of SEQ ID NO: 1.

6. A nucleic acid construct according to claim 1, wherein the open reading frame is an open reading frame selected from the group consisting of SEQ ID NO's: 1, 2, 14, and 18.

7. A nucleic acid construct according to claim 1, wherein the nucleic acid construct is an insect cell-compatible vector.

8. An insect cell comprising a nucleic acid construct according to claim 1.

9. An insect cell according to claim 8, wherein the insect cell further comprises at least one of:
i) a nucleic acid construct comprising at least one expression cassette for expression of nucleotide sequence encoding parvoviral Rep proteins; and,
ii) a nucleic acid construct comprising a transgene that is flanked by at least one parvoviral inverted terminal repeat sequence.

10. An insect cell according to claim 9, wherein at least one of the nucleic acid construct in i) and the nucleic acid construct in ii) is comprised in a baculoviral vector, or wherein the nucleic acid construct in i) is stably integrated in the genome of the insect cell.

11. A process for producing an AAV vector in an insect cell comprising the steps of:
a) culturing an insect cell as defined in claim 8 under conditions such that the AAV vector is produced; and,
b) recovery of the AAV vector,
and wherein preferably, recovery of the AAV vector in step b) comprises at least one of affinity-purification of the virion using an immobilised anti-AAV antibody, preferably a single chain camelid antibody or a fragment thereof, or filtration over a filter having a nominal pore size of 30-70 nm.

12.-15. (canceled)
